(19) United States
(12) Patent Application Publication
(10) Pub. No.: US 2026/0248873 A1
(43) Pub. Date: Aug. 27, 2026

(54) METHODS FOR ENHANCING MENTAL PERFORMANCE AND COMPOSITIONS FOR USE THEREIN

(71) Applicant: Firmenich SA, Satigny (CH)

(72) Inventors: Addi FADEL, Metuchen, NJ (US); Denis SHCHERBAKOV, Ewing, NJ (US); Nicholas O'LEARY, Pennington, NJ (US); Fabien CRAIGNOU, Satigny (CH); Corinne MONOD, Satigny (CH); Christine CHICHESTER, Satigny (CH); Christelle PORCHEROT LASSALLETTE, Eteaux (FR); Laurence CHIRAT, Satigny (CH); Arnaud STRUILLOU, ARCHAMPS (FR); Donato CEREGHETTI, Satigny (CH); Kathrin OHLA, Satigny (CH); Elisabetta RICCI, Satigny (CH); Nathalie HERBETH, Satigny (CH)

(73) Assignee: Firmenich SA, Satigny (CH)

(21) Appl. No.: 18/875,006

(22) PCT Filed: Jun. 21, 2023
(86) PCT No.: PCT/EP2023/066760

§ 371 Date: Dec. 13, 2024

Related U.S. Application Data
(60) Provisional application No. 63/354,548, filed on Jun. 22, 2022.

(30) Foreign Application Priority Data
Jul. 29, 2022 (EP) 22187945.5

(51) Int. Cl. A61K 36/898; A61K 31/09; A61K 31/122; A61K 31/365; A61K 31/366; A61K 36/18; A61K 36/48; A61K 36/63; A61K 36/738; A61K 36/752; A61K 36/82; A61P 25/28

(52) U.S. Cl. CPC A61K 36/898; A61K 31/09; A61K 31/122; A61K 31/365; A61K 31/366; A61K 36/18; A61K 36/48; A61K 36/63; A61K 36/738; A61K 36/752; A61K 36/82; A61P 25/28

(57) ABSTRACT

The present disclosure relates to methods for enhancing mental performance and compositions, typically mental performance enhancing compositions, suitable for use in such methods. The present disclosure also relates to consumer products comprising the mental performance enhancing compositions described herein.

### CROSS REFERENCE TO RELATED APPLICATIONS

The present application claims the benefit of priority of U.S. Provisional Application No. 63/354,548, filed Jun. 22, 2022, and of European Application No. 22187945.5, filed Jul. 29, 2022. The entire contents of these applications are explicitly incorporated herein by this reference.

### FIELD OF THE DISCLOSURE

The present disclosure relates to methods for enhancing mental performance and compositions, typically fragrance-containing compositions, suitable for use in such methods. The present disclosure also relates to consumer products, typically perfumed consumer products, suitable for use in methods for enhancing mental performance.

### BACKGROUND OF THE DISCLOSURE

Emotions, affective states, and other consumer benefits elicited by fragrances and aromas are important drivers of consumer satisfaction. “Odor” or “smell” usually designates the perceived sensation elicited by one or several volatile chemicals—odorants—stimulating the olfactory organ. As a result of evolution, the olfactory system has a privileged access to emotion centers of the brain, providing odors with the ability to influence the human behavior, mood, and decisions. It has been known for a long time that there is a strong link between odors and emotions (Engen, 1976). Indeed, pleasant and unpleasant odors induce differential responses at the behavioral (Bensafi, Rouby, Farget, Vigouroux, & Holley, 2002) and physiological (Bensafi, Rouby, Farget, Bertrand, et al, 2002b; Delplanque et al, 2009) levels.

However, the ability to target a particular consumer benefit, such as enhancement of cognitive function, and to select fragrance or fragrance accords to elicit such a benefit is challenging. While it is known that odors have the ability to influence human behavior, mood, and decisions, the link between odors and particular consumer benefits elicited by such odors are unclear.

Thus, there is an ongoing need for methods or processes that enable the targeting of certain consumer benefits, such as enhancement of cognitive function, particularly mental performance, including compositions, typically fragrance-containing compositions, for eliciting such benefits.

### SUMMARY OF THE DISCLOSURE

The following aspects of the present disclosure seek to address one or more of the problems described hereinabove.

In a first aspect, the present disclosure relates to a method for enhancing mental performance of a subject, the method comprising:
a) providing a mental performance enhancing composition or a consumer product comprising the said mental performance enhancing composition,
b) exposing the subject, typically a human being, to the mental performance enhancing composition or the consumer product in an amount effective to enhance the mental performance of the subject;
wherein the mental performance enhancing composition comprises one or more Musky ingredients, one or more Powdery ingredients, one or more Floral ingredients, or any combination thereof,
wherein the total amount of Musky ingredients, when present, is at least 1%, typically at least 2%, more typically at least 4%;
the total amount of Powdery ingredients, when present, is at least 0.5%, typically at least 0.6%, more typically at least 0.8%; and
the total amount of Floral ingredients, when present, is at least 10%, typically at least 15%, more typically at least 19%;
wherein the total amount of Musky ingredients, Powdery ingredients, and Floral ingredients is at least 20%, typically at least 25%, more typically at least 30%, the amounts expressed by weight relative to the total weight of the composition; in which the said Musky ingredients, Powdery ingredients, and Floral ingredients are described herein.

In a second aspect, the present disclosure relates to a mental performance enhancing composition comprising:
one or more Musky ingredients and one or more Powdery ingredients;
one or more Powdery ingredients and one or more Floral ingredients;
one or more Musky ingredients and one or more Floral ingredients; or
one or more Musky ingredients, one or more Powdery ingredients, and one or more Floral ingredients;
   wherein the total amount of Musky ingredients, when present, is at least 1%, typically at least 2%, more typically at least 4%;
   the total amount of Powdery ingredients, when present, is at least 0.5%, typically at least 0.6%, more typically at least 0.8%; and
   the total amount of Floral ingredients, when present, is at least 10%, typically at least 15%, more typically at least 19%;
   wherein the total amount of Musky ingredients, Powdery ingredients, and Floral ingredients is at least 20%, typically at least 25%, more typically at least 30%, the amounts expressed by weight relative to the total weight of the composition; in which the said Musky ingredients, Powdery ingredients, and Floral ingredients are described herein.

In a third aspect, the present disclosure relates to a consumer product, typically a perfumed consumer product, comprising the mental performance enhancing composition described herein.

In a fourth aspect, the present disclosure relates to a method for evaluating the ability of a material to deliver a consumer benefit over time, the method comprising conducting one or more sessions, wherein each session comprises:
a.) Exposing the subject to a material having an odor,
b.) Capturing the subject's perception of odor intensity,
c.) Optionally, allowing the subject to perform one or more tasks and measuring a parameter associated with each task,
d.) capturing the subject's mood or mental state,
e.) repeating steps a. to d. 1 to 10, typically 1 to 5, times.

### BRIEF DESCRIPTION OF THE FIGURES

FIG. 1 shows the accuracy (A) and response times (RTs; B, C, and D) in the Stroop task for incongruent and congruent trials and three odor conditions according to an example of the present disclosure. Boxplots represent the data from N=24; boxes range from the first to the third quartile; horizontal bars are medians; whiskers indicate 1.5 interquartile range. Points inside the boxes indicate the means; vertical bars are the standard errors. Extreme data points were omitted to increase readability of the chart.

FIG. 2 shows the accuracy (A) and RTs (B, C, and D) in the arithmetic task for easy and hard trials and three odor conditions according to an example of the present disclosure. Boxplots represent the data from N=24; boxes range from the first to the third quartile; horizontal bars are medians; whiskers indicate 1.5 interquartile range. Points inside the boxes indicate the means; vertical bars are the standard errors. Extreme data points were omitted to increase readability of the chart.

FIG. 3 shows the RTs in the Stroop task for five odor conditions according to an example of the present disclosure. Boxplots represent the data from N=60; boxes range from the first to the third quartile; horizontal bars are medians; whiskers indicate 1.5 interquartile range. Points inside the boxes indicate the means; vertical bars are the standard errors. Extreme data points were omitted to increase readability of the chart.

FIG. 4 shows the RTs in the arithmetic task for easy (A and B) and hard (C and D) trials and five odor conditions according to an example of the present disclosure. Boxplots represent the data from N=60; boxes range from the first to the third quartile; horizontal bars are medians; whiskers indicate 1.5 interquartile range. Points inside the boxes indicate the means; vertical bars are the standard errors. Extreme data points were omitted to increase readability of the chart.

FIG. 5 shows the accuracy (A), RTs (B) in the Stroop task and subjective focus (C) for five repetitions according to an example of the present disclosure. Points indicate the means; vertical bars are the standard errors.

FIG. 6 shows the accuracy in the Stroop task for three odor conditions according to an example of the present disclosure. Bars indicate the means; vertical lines are the standard errors.

FIG. 7 shows subjective focus for five repetitions and three odor conditions according to an example of the present disclosure. Points indicate the means; vertical bars are the standard errors.

### DETAILED DESCRIPTION

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

Unless defined otherwise, all technical and scientific terms used herein have the same meaning as is commonly understood by one of skill in the art to which this specification pertains.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

Throughout the present disclosure, various chemical names and structures may be recited. Unless otherwise stated, any stereoisomers, such as enantiomers, diastereomers, anomers, epimers, and the like; and geometric isomers, such as cis/trans or E/Z isomers, of the recited chemical name or structure are contemplated. As would be understood by those of ordinary skill in the art, stereoisomers may possess one stereocenter, giving rise to enantiomers, or more than one stereocenter, giving rise to diastereomers, each stereocenter having one of two different stereochemistries (i.e., R or S). Enantiomers may be characterized by their ability to rotate oncoming plane-polarized light to the right, designated as dextrorotatory, “(+)” or “D”, or to the left, designated as levorotatory, “(−)” or “L”. Enantiomers may exist as racemic mixtures or scalemic mixtures. Geometric isomers refer to isomers in which the spatial relationship of atoms around a double bond are different, typically designated E or Z according to conventional understanding in the chemical art. Geometric isomers may also exist as mixtures of E and Z isomers. All of the aforementioned isomeric variations of the chemical names or structures recited herein are included.

In the first aspect, the present disclosure relates to a method for enhancing mental performance of a subject, the method comprising:
a) providing a mental performance enhancing composition or a consumer product comprising the said mental performance enhancing composition,
b) exposing the subject, typically a human being, to the mental performance enhancing composition or the consumer product in an amount effective to enhance the mental performance of the subject;
wherein the mental performance enhancing composition comprises one or more Musky ingredients, one or more Powdery ingredients, one or more Floral ingredients, or any combination thereof,
wherein the total amount of Musky ingredients, when present, is at least 1%, typically at least 2%, more typically at least 4%;
the total amount of Powdery ingredients, when present, is at least 0.5%, typically at least 0.6%, more typically at least 0.8%; and
the total amount of Floral ingredients, when present, is at least 10%, typically at least 15%, more typically at least 19%;
wherein the total amount of Musky ingredients, Powdery ingredients, and Floral ingredients is at least 20%, typically at least 25%, more typically at least 30%, the amounts expressed by weight relative to the total weight of the composition; in which the said Musky ingredients, Powdery ingredients, and Floral ingredients are described herein.

It has been discovered that mental performance enhancing compositions comprising one or more Musky ingredients, one or more Powdery ingredients, one or more Floral ingredients, or any combination thereof, can enhance the mental performance of a subject, typically a human being. The mental performance enhancing compositions described herein may be fragrance compositions as the one or more Musky ingredients, one or more Powdery ingredients, one or more Floral ingredients, or any combination thereof, are ingredients suitable for use in fragrance compositions.

Exposing the subject, typically a human being, to the mental performance enhancing composition or the consumer product in an amount effective to enhance the mental performance of the subject may be accomplished using any method known to those of ordinary skill in the art capable of delivering an odor to the nose of a subject.

The method for enhancing mental performance of a subject described herein is a non-therapeutic method and does not relate to the treatment of any disease or curing of any disease or illness. The method described herein provides a positive consumer benefit in the form of enhancement of mental performance.

In some embodiments, enhancement of mental performance may be an increase of mental processing speed or efficiency, or a decrease in reaction time in performing a cognitive task, such as, for example, a Stroop task, a mental arithmetic task, and the like. A Stroop task is a classical paradigm to measure interference and attention (see, for example, Stroop, J. R. (1935). Studies of interference in serial verbal reactions. Journal of Experimental Psychology, 18, 643-662 and MacLeod, C. M. (1991) Half a Century of Research on the Stroop Effect: An Integrative Review. Psychological Bulletin, 109 (2), 163-203). A mental arithmetic task requires quantitative reasoning, working memory and decision making in an ecologically valid setting (see, for example, Wechsler, D. (2014). Wechsler intelligence scale for children-fifth edition. Bloomington, MN: Pearson and Wechsler, D. (2008). Wechsler Adult Intelligence Scale—Fourth Edition (WAIS-IV) [Database record]. APA PsycTests.)

In some embodiments, the subject's reaction time in performing a task, typically a Stroop task and/or mental arithmetic task, is reduced during or after exposure to the mental performance enhancing composition or a consumer product comprising the said mental performance enhancing composition.

In some embodiments, the subject's accuracy in performing a task, typically a Stroop task and/or mental arithmetic task, is increased during or after exposure to the mental performance enhancing composition or a consumer product comprising the said mental performance enhancing composition.

In some embodiments, the subject's subjective focus, or feelings of being focused, as measured with a questionnaire, while performing a task, typically a Stroop task and/or mental arithmetic task, is increased during or after exposure to the mental performance enhancing composition or a consumer product comprising the said mental performance enhancing composition.

In some embodiments, the enhancement of mental performance is sustained over a period of at least 2 hours.

Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE,
(+−)-1-(3,5,5,6,8,8-hexamethyl-5,6,7,8-tetrahydro-2-naphthalenyl)ethenone,
(+−)-(4E)-3-METHYL-4-CYCLOPENTADECEN-1-ONE,
(+−)-3-METHYL-5-CYCLOPENTADECEN-1-ONE,
2-{(1 RS)-1-[3,3-dimethylcyclohexyl]ethoxy}-2-oxoethyl propionate,
2-oxo-2-{[2,6,6-trimethylcycloheptyl]oxy}ethyl propionate,
(+−)-4,6,6,7,8,8-hexamethyl-1,3,4,6,7,8-hexahydrocyclopenta[g]isochromene,
1-OXA-12-CYCLOHEXADECEN-2-ONE,
1-OXA-13-CYCLOHEXADECEN-2-ONE,
(9Z)-9-cycloheptadecen-1-one,
CYCLOPENTADECANONE,
oxacyclohexadecan-2-one,
1,4-dioxacycloheptadecane-5,17-dione,
(10E)-oxacycloheptadec-10-en-2-one,
(Z)-4-CYCLOPENTADECEN-1-ONE,
(6RS, 7RS)-3,5,5,6,7,8,8-heptamethyl-5,6,7,8-tetrahydro-2-naphthalenecarbaldehyde,
1,4-dioxacyclohexadecane-5,16-dione,
(1S)-1-[(1R)-3,3-dimethylcyclohexyl]ethyl [3-ethyl-2-oxiranyl]acetate,
(+−)-3-methylcyclopentadecanone,
AMBRETTE SEED ABSOLUTE, and any combination thereof.

Powdery ingredients are selected from the group consisting of:
TONKA ABSOLUTE,
VANILLA BOURBON,
VANILLE TAHITENSIS,
(3RS,3aRS,6SR,7aSR)-3,6-dimethylhexahydro-1-benzofuran-2(3H)-one,
(3RS,3aSR,6RS,7aRS)-3,6-dimethylhexahydro-1-benzofuran-2(3H)-one,
2-ETHOXY-4-METHYLPHENOL,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde,
2-CHROMENONE,
2-methoxy-4-methylphenyl methyl carbonate,
3,4-dimethoxybenzaldehyde,
4-(4-hydroxyphenyl)-2-butanone,
1,4-dimethoxybenzene,
(1 RS,2RS,7SR,8RS,9Z)-9-ethylidene-3-oxatricyclo[6.2.1.0~2,7~]undecan-4-one,
(1 RS,2RS,7SR,8SR,10Z)-10-ethylidene-3-oxatricyclo[6.2.1.0~2,7~]undecan-4-one,
(1 RS,2RS,7SR,8RS,9E)-9-ethylidene-3-oxatricyclo[6.2.1.0~2,7~]undecan-4-one,
(1 RS,2SR,7SR,8SR,10E)-10-ethylidene-3-oxatricyclo[6.2.1.0~2,7~]undecan-4-one,
FOIN ABS,
(+−)-4-hexanolide,
LIATRIX ABS TYPE,
4-(1,3-benzodioxol-5-yl)-2-butanone,
4-(4-hydroxyphenyl)-2-butanone,
VANILLE MADAGASCAR SFE 25 TEC,
VANILLE SURABSOLUE INCOLORE,
4-hydroxy-3-methoxybenzaldehyde,
and any combination thereof.

Floral ingredients are selected from the group consisting of:
METHYL N-(7-HYDROXY-3,7-DIMETHYL-1-OCTENYL)ANTHRANILATE,
CANANGA OIL,
(2E,6E)-3,7,11-trimethyl-2,6,10-dodecatrien-1-ol,
FLEUR D ORANGER ABS,
ORANGE FLOWERS,
methyl 3,7-dimethyl-2,6-octadienoate,
GERANIUM ROSE OIL,
IRIS RESINOIDE,
JASMIN EGYPTE INFUSION,
JASMIN SAMBAC ABS CHINE PERF,
JASMINE OIL,
JASMIN INDES ABS SUPER,
METHYL N-[3-(4-TERT-BUTYLPHENYL)-2-METHYL-1-PROPENYL]ANTHRANILATE,
LILAC FIRBEST,
methyl 2-({[3-(4-hydroxy-4-methylpentyl)-3-cyclohexen-1-ylidene]methyl}amino)benzoate,
methyl 2-({[4-(4-hydroxy-4-methylpentyl)-3-cyclohexen-1-ylidene]methyl}amino)benzoate,
MICHELIA ALBA OIL,
MATE ABS,
MIMOSA PAYS ABS,
NARCISSUS ABS,
C6-12 ETHOXYLATED ALCOHOLS,
NEROLI,
OSMANTHUS ABS NAT,
PETITGRAIN ITALY EO,
PETITGRAIN S TERP,
ROSE ABS BULGARE,
TEA BLACK CEYLAN EXTR,
(+−)-2-propylheptanenitrile,
(2E)-2-METHYL-3-(4-METHYLPHENYL)-2-PROPEN-1-OL,
TUBEROSE INDIA ABS,
YLANG VOP,
1-(3-METHYL-1-BENZOFURAN-2-YL)ETHANONE,
1-(4-methylphenyl)ethenone,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
2-HEXYL-2-CYCLOPENTEN-1-ONE,
3-(4-TERT-BUTYLPHENYL)PROPANAL,
3,7-DIMETHYL-6-OCTEN-1-OL,
3,7-DIMETHYL-7-OCTEN-1-OL,
(+−)-2,4-dimethyl-4,4a,5,9b-tetrahydroindeno[1,2-d][1,3]dioxine,
ETHYL BENZOATE,
ETHYL PHENYLACETATE,
(+−)-(E)-3-METHYL-4-(2,6,6-TRIMETHYL-2-CYCLOHEXEN-1-YL)-3-BUTEN-2-ONE,
2-PHENYLETHANOL,
4-methyl-2-(2-methyl-1-propen-1-yl)tetrahydro-2H-pyran,
4-methyl-2-phenyltetrahydro-2H-pyran,
(3E)-4-(2,6,6-trimethyl-1-cyclohexen-1-yl)-3-buten-2-one,
(−)-(2S,4R)-4-methyl-2-(2-methyl-1-propen-1-yl)tetrahydro-2H-pyran,
1-(2-naphthyl)ethenone,
(4E)-4-methyl-5-(4-methylphenyl)-4-pentenal,
TRICYCLO[5.2.1.0(2,6)]DEC-3-EN-8-YL PROPANOATE,
3-methyl-2-[(2Z)-2-penten-1-yl]-2-cyclopenten-1-one,
(+−)-(3E)-4-(2,6,6-trimethyl-2-cyclohexen-1-yl)-3-buten-2-one,
(3E)-4-(2,6,6-trimethyl-1-cyclohexen-1-yl)-3-buten-2-one,
(1E)-1-(2,6,6-trimethyl-1-cyclohexen-1-yl)-1-penten-3-one,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
1-METHOXY-4-METHYLBENZENE,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-3-methyl-5-phenyl-1-pentanol,
(+−)-3,7-dimethyl-1,6-octadien-3-ol,
(+−)-(E)-3-METHYL-4-(2,6,6-TRIMETHYL-2-CYCLOHEXEN-1-YL)-3-BUTEN-2-ONE,
(E)-1-(2,6,6-TRIMETHYL-2-CYCLOHEXEN-1-YL)-1-PENTEN-3-ONE,
(+−)-(E)-1-(2,2-DIMETHYL-6-METHYLENE-1-CYCLOHEXYL)-1-PENTEN-3-ONE,
(E)-1-(2,6,6-TRIMETHYL-1-CYCLOHEXEN-1-YL)-1-PENTEN-3-ONE,
4-methyl-6-phenyl-2-hexanol,
3,7-DIMETHYL-2,6-OCTADIEN-1-OL,
(+−)-3-(1,3-BENZODIOXOL-5-YL)-2-METHYLPROPANAL,
(+−)-(E)-TRANS-alpha-IRONE,
(+−)-(E)-CIS-alpha-IRONE,
(+−)-(E)-beta-IRONE,
3,7-DIMETHYL-1,6-NONADIEN-3-OL,
methyl [3-oxo-2-pentylcyclopentyl]acetate,
2-PHENYLETHYL ACETATE,
2-PHENYLETHYL FORMATE,
(3Z)-3-hexen-1-yl salicylate,
1,1′-oxydibenzene,
tricyclo[5.2.1.0~2,6-]dec-3-en-8-yl acetate,
tricyclo[5.2.1.0~2,6-]dec-4-en-8-yl acetate,
(+−)-TRANS-3-METHYL-4-NONANOLIDE,
(+−)-3,7-DIMETHYL-3-OCTANOL,
(+−)-(Z)-7-DECEN-4-OLIDE,
(+−)-3,7-DIMETHYL-6-OCTEN-1-OL,
2-PHENYLETHYL PHENYLACETATE,
(+−)-5-ETHYL-2-NONANOL,
(+)-METHYL (1R)-CIS-3-OXO-2-PENTYL-1-CYCLOPENTANEACETATE,
1-octanol,
benzyl acetate,
CYCLOHEXYL 2-HYDROXYBENZOATE,
(+−)-8-DECEN-5-OLIDE,
CYCLOHEXYLIDENE(PHENYL)ACETONITRILE,
(+−)-3-(4-isopropylphenyl)-2-methylpropanal,
METHYL BENZOATE,
2-pentylcyclopentanol,
7-HYDROXY-3,7-DIMETHYLOCTANAL,
(+−)-7-hydroxy-3,7-dimethyloctanal,
(+−)-(3-methoxy-2-methylpropyl)benzene,
(+−)-3,7-dimethyl-1-octen-3-ol,
methyl cyclopentylideneacetate,
2-PHENYLETHYL 2-HYDROXYBENZOATE,
1-(5,6,7,8-tetrahydro-2-naphthalenyl)ethenone,
(+−)-2,2,2-TRICHLORO-1-PHENYLETHYL ACETATE,
methyl 2-((1 RS,2RS)-3-oxo-2-pentylcyclopentyl)acetate,
(2Z)-2-phenyl-2-hexenenitrile,
1,3-NONANEDIYL DIACETATE,
TETRAHYDRO-3-PENTYL-4(2H)-PYRANYL ACETATE,
(+−)-3,7-DIMETHYL-6-OCTENYL PROPANOATE,
(Z)-3,7-DIMETHYL-2,6-OCTADIEN-1-OL,
2-ETHOXYNAPHTHALENE,
(E)-2-PENTYL-3-PHENYL-2-PROPENAL,
9-DECEN-1-OL,
2-PHENYLETHYL ISOBUTYRATE,
benzyl salicylate,
4-CYCLOHEXYL-2-METHYL-2-BUTANOL,
1-NONANOL,
3-METHYLBUTYL 2-PHENYLETHYL ETHER,
(+−)-3,7-dimethyl-3-octanyl acetate,
ISOBUTYL PHENYLACETATE,
methyl {(1RS,2RS)-3-oxo-2-[(2Z)-2-penten-1-yl]cyclopentyl}acetate,
(+−)-TETRAHYDRO-2-ISOBUTYL-4-METHYL-4(2H)-PYRANOL,
ISOBUTYL 2-HYDROXYBENZOATE,
(−)-(2S)-1-oxo-1-(2-propanyloxy)-2-propanyl 2,2-dimethylpropanoate,
1-dodecanol,
(+−)-2-phenyl-1-propanol,
2-METHYLBUTYL SALICYLATE,
PENTYL SALICYLATE,
3-METHYLBUTYL PHENYLACETATE,
2-METHYLBUTYL PHENYLACETATE,
3-pentyltetrahydro-2H-pyran-4-yl acetate,
1,1-DIMETHYL-2-PHENYLETHYL ACETATE,
3,7-DIMETHYL-6-OCTENYL ACETATE,
BENZYL PHENYLACETATE,
[trans-4-(2-propanyl)cyclohexyl]methanol,
[cis-4-(2-propanyl)cyclohexyl]methanol,
(2E)-2-benzylideneoctanal,
2-BENZYL-4,4,6-TRIMETHYL-1,3-DIOXANE,
3-[(1R)-4-methyl-3-cyclohexen-1-yl]-1-butanol,
CIS-2-BENZYL-1,3-DIOXAN-5-OL,
2-BENZYL-1,3-DIOXOLANE-4-METHANOL,
TRANS-2-BENZYL-1,3-DIOXAN-5-OL,
6,10-dimethyl-5,9-undecadien-2-one,
HEXYL 2-HYDROXYBENZOATE,
3,7,11-TRIMETHYL-1,6,10-DODECATRIEN-3-OL,
2-phenoxyethanol,
1-PHENYL-1-ETHANOL,
(E)-3,7-DIMETHYL-2,6-OCTADIENYL PROPANOATE,
2-METHYL-4-PHENYL-2-BUTANOL,
(+−)-2,4,6-TRIMETHYL-3-CYCLOHEXENE-1-METHANOL,
2-METHYL-1-PHENYL-2-PROPANOL,
2-phenoxyethyl 2-methylpropanoate,
decyl acetate,
(E)-3,7-dimethyl-2,6-octadienyl acetate,
(2Z)-3,7-dimethyl-2,6-octadien-1-yl acetate,
(+−)-3,7-dimethyloctyl acetate,
tricyclo[5.2.1.0~2,6-]dec-3-en-8-yl acetate,
tricyclo[5.2.1.0~2,6-]dec-4-en-8-yl acetate,
citronellyl acetate,
neryl acetate,
geranyl acetate,
3,5,5-trimethylhexyl acetate,
4-phenyl-2-butanone,
4-(2,6,6-trimethyl-1-cyclohexen-1-yl)-2-butanone,
(3E)-4-(2,6,6-trimethyl-1-cyclohexen-1-yl)-3-buten-2-one,
4-(1,3-benzodioxol-5-yl)-2-butanone,
CAPUCINE M SA,
CASSIE ABSOLUTE,
1-phenyl-2-(1-propoxyethoxy)ethane,
(+−)-8,8-dimethyl-1,2,3,4,5,6,7,8-octahydro-2-naphthalenecarbaldehyde,
(+−)-5,5-dimethyl-1,2,3,4,5,6,7,8-octahydro-2-naphthalenecarbaldehyde,
(+−)-2,2-dimethyltricyclo[6.2.1.0~1,6-]undecan-7-one,
(+−)-2-pentylcyclopentanone,
2,6-dimethyl-2-heptanol,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
(+−)-(4Z)-4,8-dimethyl-4,9-decadienal,
(+−)-(4E)-4,8-dimethyl-4,9-decadienal,
(+−)-3-(3-isopropyl-1-phenyl)butanal,
(+−)-3,7-dimethyl-6-octenyl formate,
(+−)-3,7-dimethyl-6-octen-1-yl formate,
(2E)-3,7-dimethyl-2,6-octadien-1-yl formate,
GARDENIA FIRABSOLUE,
thiodamascone,
(1Z)-2-methyl-1-undecen-1-yl 2-phenylethyl ether,
(1E)-2-methyl-1-undecen-1-yl 2-phenylethyl ether,
thioionone,
(+−)-4-(dodecylthio)-4-(2,6,6-trimethyl-2-cyclohexen-1-yl)-2-butanone,
(+−)-4-(dodecylthio)-4-(2,6,6-trimethyl-1-cyclohexen-1-yl)-2-butanone,
benzo[d][1,3]dioxole-5-carbaldehyde,
(+−)-(3E)-4-(2,6,6-trimethyl-2-cyclohexen-1-yl)-3-buten-2-one,
2-nonenenitrile,
cis-4-(2-methyl-2-propanyl)cyclohexyl acetate,
trans-4-(2-methyl-2-propanyl)cyclohexyl acetate,
MUGUET FIRBEST,
(+−)-(4Z)-5-cyclohexyl-2,4-dimethyl-4-pentenal,
(+−)-(4E)-5-cyclohexyl-2,4-dimethyl-4-pentenal,
ORENOL P SAD,
PALMAROSA,
PIVOINE BLANCHE FIRBEST,
benzyl propanoate,
(+−)-methyl 2,2-dimethyl-6-methylidenecyclohexanecarboxylate,
(−)-(3R)-3,7-dimethyl-1,6-octadien-3-ol,
(+−)-3,7-dimethyl-1-octanol,
TEA GREEN FIRBEST,
3-(4,4-dimethyl-1-cyclohexen-1-yl)propanal,
LINDEN FIRBEST,
VIOTRIL BASE,
PETITGRAIN PARAGUAY HE,
YLANG EXTRA INFUSION,
YLANG EXTRA FIRNAT,
3-[4-(2-hydroxy-2-methylpropyl)phenyl]propanal,
(+−)-2-[4-(2-hydroxy-2-methylpropyl)phenyl]propanal,
3-(3-(2-hydroxy-2-methylpropyl)phenyl)propanal,
and any combination thereof.

The total amount of Musky ingredients, when present, is at least 1%, typically at least 2%, more typically at least 4%. In some embodiments, the total amount of Musky ingredients, when present, is from 1% to 95%, typically from 2% to 80%, more typically from 4% to 70%. In some embodiments, the total amount of Musky ingredients, when present, is from 5% to 50%, typically from 10% to 40%, more typically from 15% to 30%, by weight relative to the total weight of the composition.

The total amount of Powdery ingredients, when present, is at least 0.5%, typically at least 0.6%, more typically at least 0.8%. In some embodiments, the total amount of Powdery ingredients, when present, is from 0.5% to 95%, typically from 0.6% to 80%, more typically from 0.8% to 70%. In some embodiments, the total amount of Powdery ingredients, when present, is from 0.5% to 20%, typically from 0.6% to 10%, more typically from 0.8% to 5%, by weight relative to the total weight of the composition.

The total amount of Floral ingredients, when present, is at least 10%, typically at least 15%, more typically at least 19%. In some embodiments, the total amount of Floral ingredients, when present, is from 10% to 95%, typically from 15% to 80, more typically from 19% to 70%.

The total amount of Musky ingredients, Powdery ingredients, and Floral ingredients is at least 20%, typically at least 25%, more typically at least 30%, the amounts expressed by weight relative to the total weight of the composition. In some embodiments, the total amount of Musky ingredients, Powdery ingredients, and Floral ingredients is from 20% to 95%, typically from 25% to 80%, more typically from 30% to 70%.

In an embodiment, the mental performance enhancing composition comprises:
one or more Musky ingredients and one or more Powdery ingredients;
one or more Powdery ingredients and one or more Floral ingredients;
one or more Musky ingredients and one or more Floral ingredients; or
one or more Musky ingredients, one or more Powdery ingredients, and one or more Floral ingredients. In an embodiment, the mental performance enhancing composition comprises one or more Musky ingredients and one or more Powdery ingredients. In another embodiment, the mental performance enhancing composition comprises one or more Powdery ingredients and one or more Floral ingredients. In yet another embodiment, the mental performance enhancing composition comprises one or more Musky ingredients, one or more Powdery ingredients, and one or more Floral ingredients.

Of the Musky, Powdery, and Floral ingredients described herein, certain Musky, Powdery, and Floral ingredients are especially useful in the mental performance enhancing composition.

Thus, in some embodiments, the one or more Musky ingredients are selected from the group consisting of:
(6RS,7RS)-3,5,5,6,7,8,8-heptamethyl-5,6,7,8-tetrahydro-2-naphthalenecarbaldehyde and
(1S)-1-[(1R)-3,3-dimethylcyclohexyl]ethyl [3-ethyl-2-oxiranyl]acetate.

In some embodiments, the one or more Powdery ingredients are selected from the group consisting of:
VANILLE TAHITENSIS,
(3S,3aS,6R,7aR)-3,6-dimethylhexahydro-1-benzofuran-2(3H)-one,
1-oxaspiro[4.5]decan-2-one,
2-methoxy-4-methylphenyl methyl carbonate,
VANILLE MADAGASCAR SFE 25 TEC,
VANILLE SURABSOLUE INCOLORE,
4-hydroxy-3-methoxybenzaldehyde,
and any combination thereof.

In some embodiments, the one or more Floral ingredients are selected from the group consisting of:
JASMIN EGYPTE INFUSION,
LILAC FIRBEST,
PETITGRAIN S TERP,
(+−)-2-propylheptanenitrile,
(2E)-2-METHYL-3-(4-METHYLPHENYL)-2-PROPEN-1-OL,
(4E)-4-methyl-5-(4-methylphenyl)-4-pentenal,
4-methyl-6-phenyl-2-hexanol,
(+−)-(Z)-7-DECEN-4-OLIDE,
(+)-METHYL (1R)-CIS-3-OXO-2-PENTYL-1-CYCLOPENTANEACETATE,
(−)-(2S)-1-oxo-1-(2-propanyloxy)-2-propanyl 2,2-dimethylpropanoate,
YLANG EXTRA INFUSION,
YLANG EXTRA FIRNAT,
or any combination thereof.

In some instances, certain Musky, Powdery, and Floral ingredients described herein have been found to be especially impactful when used according to the method, composition, and/or consumer product of the present disclosure.

Thus, in an embodiment, the total amount of the Musky ingredients selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE,
(+−)-(4E)-3-METHYL-4-CYCLOPENTADECEN-1-ONE,
(+−)-3-METHYL-5-CYCLOPENTADECEN-1-ONE,
2-{(1 RS)-1-[3,3-dimethylcyclohexyl]ethoxy}-2-oxoethyl propionate,
2-oxo-2-{[2,6,6-trimethylcycloheptyl]oxy}ethyl propionate,
(+−)-4,6,6,7,8,8-hexamethyl-1,3,4,6,7,8-hexahydrocyclopenta[g]isochromene,
1-OXA-12-CYCLOHEXADECEN-2-ONE,
1-OXA-13-CYCLOHEXADECEN-2-ONE,
oxacyclohexadecan-2-one,
1,4-dioxacycloheptadecane-5,17-dione,
(10E)-oxacycloheptadec-10-en-2-one, and any combination thereof;

the Powdery ingredients selected from the group consisting of:
3,6-dimethylhexahydro-1-benzofuran-2(3H)-one,
3-ethoxy-4-hydroxybenzaldehyde,
2-CHROMENONE,
and any combination thereof; and

the Floral ingredients selected from the group consisting of:
ROSE ABS BULGARE,
(+−)-2-propylheptanenitrile,
(2E)-2-METHYL-3-(4-METHYLPHENYL)-2-PROPEN-1-OL,
(+−)-2-methyl-3-[4-(2-methyl-2-propanyl)phenyl]propanal,
3-(4-TERT-BUTYLPHENYL)PROPANAL,
(+−)-(E)-3-METHYL-4-(2,6,6-TRIMETHYL-2-CYCLOHEXEN-1-YL)-3-BUTEN-2-ONE,
2-PHENYLETHANOL,
4-methyl-2-(2-methyl-1-propen-1-yl)tetrahydro-2H-pyran,
1-(2-naphthyl)ethenone,
(4E)-4-methyl-5-(4-methylphenyl)-4-pentenal,
TRICYCLO[5.2.1.0(2,6)]DEC-3-EN-8-YL PROPANOATE,
TRICYCLO[5.2.1.0(2,6)]DEC-4-EN-8-YL PROPANOATE,
3-methyl-2-[(2Z)-2-penten-1-yl]-2-cyclopenten-1-one,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-3-methyl-5-phenyl-1-pentanol,
(+−)-3-(1,3-BENZODIOXOL-5-YL)-2-METHYLPROPANAL,
(+−)-(E)-TRANS-alpha-IRONE,
(+−)-(E)-CIS-alpha-IRONE,
(+−)-(E)-beta-IRONE,
3,7-DIMETHYL-1,6-NONADIEN-3-OL,
methyl [3-oxo-2-pentylcyclopentyl]acetate,
(3Z)-3-hexen-1-yl salicylate,
(+−)-3,7-DIMETHYL-3-OCTANOL,
(+−)-3,7-DIMETHYL-6-OCTEN-1-OL,
(+)-METHYL (1R)-CIS-3-OXO-2-PENTYL-1-CYCLOPENTANEACETATE,
benzyl acetate,
(+−)-8-DECEN-5-OLIDE,
CYCLOHEXYLIDENE(PHENYL)ACETONITRILE,
(+−)-3-(4-isopropylphenyl)-2-methylpropanal,
(+−)-7-hydroxy-3,7-dimethyloctanal,
(+−)-2,2,2-TRICHLORO-1-PHENYLETHYL ACETATE,
methyl 2-((1 RS,2RS)-3-oxo-2-pentylcyclopentyl)acetate,
2-ETHOXYNAPHTHALENE,
2-PHENYLETHYL ISOBUTYRATE,
benzyl salicylate,
4-CYCLOHEXYL-2-METHYL-2-BUTANOL,
1-NONANOL,
(+−)-TETRAHYDRO-2-ISOBUTYL-4-METHYL-4(2H)-PYRANOL,
(−)-(2S)-1-oxo-1-(2-propanyloxy)-2-propanyl 2,2-dimethylpropanoate,
1,1-DIMETHYL-2-PHENYLETHYL ACETATE,
3,7-DIMETHYL-6-OCTENYL ACETATE,
[4-(2-propanyl)cyclohexyl]methanol,
(2E)-2-benzylideneoctanal,
3-[(1R)-4-methyl-3-cyclohexen-1-yl]-1-butanol,
HEXYL 2-HYDROXYBENZOATE,
3,7,11-TRIMETHYL-1,6,10-DODECATRIEN-3-OL,
1-PHENYL-1-ETHANOL,
2-METHYL-1-PHENYL-2-PROPANOL,
and any combination thereof;
   is at least 5%, typically at least 7%, more typically at least 10%, by weight relative to the total weight of the composition.

In an embodiment, the one or more Floral ingredients are selected from the group consisting of:
(+−)-2-propylheptanenitrile,
(2E)-2-METHYL-3-(4-METHYLPHENYL)-2-PROPEN-1-OL,
(4E)-4-methyl-5-(4-methylphenyl)-4-pentenal,
(+)-METHYL (1R)-CIS-3-OXO-2-PENTYL-1-CYCLOPENTANEACETATE,
(−)-(2S)-1-oxo-1-(2-propanyloxy)-2-propanyl 2,2-dimethylpropanoate,
or a combination thereof.

In an embodiment, the total amount of the Musky ingredients, the Powdery ingredients, and the Floral ingredients is from 5% to 95% typically from 7% to 80%, more typically 10% to 70%, by weight relative to the total weight of the composition.

The total number of Musky ingredients, Powdery ingredients, and Floral ingredients used according to the method, composition, and/or consumer product of the present disclosure is not particularly limited. However, in an embodiment, the total number of Musky ingredients, Powdery ingredients, and Floral ingredients is at least 3, typically at least 5, more typically at least 7.

In another embodiment, the total number of Musky ingredients, Powdery ingredients, and Floral ingredients is from 3 to 20, typically from 5 to 15, more typically from 7 to 10.

In some instances, certain Musky, Powdery, and/or Floral ingredients described herein have been found to be high performers with respect to providing sustained, long lasting trail performance in addition to providing the desired consumer benefit.

Thus, in an embodiment, the one or more Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE], and any combination thereof;

the one or more Powdery ingredients are selected from the group consisting of:
3,6-dimethylhexahydro-1-benzofuran-2(3H)-one,
2-ETHOXY-4-METHYLPHENOL,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde, and any combination thereof;

the one or more Floral ingredients are selected from the group consisting of:
1-(3-METHYL-1-BENZOFURAN-2-YL)ETHANONE,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
3-(4-TERT-BUTYLPHENYL)PROPANAL,
3,7-DIMETHYL-6-OCTEN-1-OL,
3,7-DIMETHYL-7-OCTEN-1-OL,
(+−)-2,4-dimethyl-4,4a,5,9b-tetrahydroindeno[1,2-d][1,3]dioxine, and any combination thereof.

In another embodiment, the one or more Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE], and any combination thereof;

the one or more Powdery ingredients are selected from the group consisting of:
3,6-dimethylhexahydro-1-benzofuran-2(3H)-one,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde, and any combination thereof;

the one or more Floral ingredients are selected from the group consisting of:
1-(3-METHYL-1-BENZOFURAN-2-YL)ETHANONE,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
3-(4-TERT-BUTYLPHENYL)PROPANAL,
3,7-DIMETHYL-6-OCTEN-1-OL,
3,7-DIMETHYL-7-OCTEN-1-OL,
(+−)-2,4-dimethyl-4,4a,5,9b-tetrahydroindeno[1,2-d][1,3]dioxine, and any combination thereof.

The mental performance enhancing composition may further comprise at least one ingredient, typically at least two ingredients, more typically at least 3 ingredients, that are not categorized as being Musky, Powdery, or Floral, but provide olfactive notes close to Musky, Powdery, or Floral and have been found to help create a hedonically faceted note without disrupting the main olfactive direction described herein. Such ingredients include, but are not limited to:
2,2,9,11-tetramethylspiro[5.5]undec-8-en-1-yl acetate,
7-PROPYL-2H,4H-1,5-BENZODIOXEPIN-3-ONE,
(+−)-1-(5-propyl-1,3-benzodioxol-2-yl)ethenone,
(3aRS,5aSR,9aSR,9bSR)-3a,6,6,9a-tetramethyldodecahydronaphtho[2,1-b]furan,
13-oxabicyclo[10.1.0]trideca-4,8-diene,
CLEARWOOD PRISMA CONTAINING PATCHOULOL,
(3S,3aR,5R,8S,8aS)-5-isopropenyl-3,8-dimethyloctahydro-3a(1H)-azulenol,
7-isopropenyl-1,4-dimethyldecahydro-4-azulenol,
(1R,3R,6S,7S,8S)-2,2,6,8-tetramethyltricyclo[5.3.1.0~3,8-]undecan-3-ol,
(1R,3R,6S,7S,8S)-3-ethoxy-2,2,6,8-tetramethyltricyclo[5.3.1.0~3,8-]undecane,
(+−)-6-METHYL-7-OXA-1-THIA-4-AZASPIRO[4.4]NONANE,
(+−)-(1-ethoxyethoxy)cyclododecane,
(3RS)-1-[2,2,3,6-tetramethylcyclohexyl]-3-hexanol,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
(3E)-4-[2,2,3,6-tetramethylcyclohexyl]-3-buten-2-one,
2-methyl-4-[(1R)-2,2,3-trimethyl-3-cyclopenten-1-yl]-4-pentenal,
(+)-3,3-dimethyl-5-[(1 S)-2,2,3-trimethyl-3-cyclopenten-1-yl]-4-penten-2-ol
(+−)-3ENDO-METHOXY-7,7-DIMETHYL-10-METHYLENE-BICYCLO[4.3.1]DECANE,
(+−)-3EXO-METHOXY-7,7-DIMETHYL-10-METHYLENE-BICYCLO[4.3.1]DECANE, 1-decen-4-yne,
PATCHOULOL CRIST 50 HED,
(2RS,4aRS,8aSR)-5,5,8a-trimethyldecahydro-2-naphthalenyl acetate,
2,6,10,10-tetramethyl-1-oxaspiro[4.5]decan-6-ol,
4-ethyl-8-methyloctahydronaphthalen-1(2H)-one,
(+)-(1 S,2S,3S,5R)-2,6,6-trimethylspiro[bicyclo[3.1.1]heptane-3,1′-cyclohexane]-2′-en-4′-one,
3-PROPYLPHENOL,
5,5,9,13-tetramethyl-14,16-dioxatetracyclo[11.2.1.0~1,10-.0-4,9-]hexadecane, and
any combination thereof.

The mental performance enhancing composition may further comprise a phytoactive enhancer. In general, phytoactive compounds are obtained from plant sources, such as for example, a wide variety of algae, bryophytes, pteridophytes, gymnosperms, angiosperms, and the like. Certain phytoactive compounds or oils may aid in the enhancement of mental performance when used according to the method, composition, and/or consumer product of the present disclosure. Exemplary phytoactive enhancers include, but are not limited to, compounds or oils originating from Vetivera Zizanioides, Lavandula Angustifolia, Frereana, Boswellia Carterii, Boswellia Sacra, Santalum Album, Leptospermum Scoparium, Origanum Majorana, Elettariia Cardamomum, Jasminum Grandiflorum, Pelargonium Graveolens, Citrus Bergamia, Helichrysum Italicum, and the like.

It has been discovered that certain ingredients disrupt the ability of the mental performance enhancing compositions according to the present disclosure to provide enhancement of mental performance in a subject. Aromatic ingredients, Spicy ingredients, Citrus ingredients, or any combination thereof, are considered disruptors and the presence of such ingredients should be limited.

In an embodiment, the mental performance enhancing composition comprises:
from 0% to 5.5%, typically from 0% to 5%, more typically from 0% to 4.5%, of Aromatic ingredients,
from 0% to 3.5%, typically from 0% to 3%, more typically from 0% to 1% of Spicy ingredients,
from 0% to 5.5%, typically from 0% to 5%, more typically from 0% to 4.5%, of Citrus ingredients, or any combination thereof, wherein the total amount of Aromatic ingredients, Spicy ingredients, and Citrus ingredients is from 0% to 10%, typically from 0% to 6%, the amounts expressed by weight relative to the total weight of the composition.

In an embodiment, the mental performance enhancing composition comprises:
from 0% to 1%, typically from 0% to 0.7%, more typically from 0% to 0.4%, of Aromatic ingredients,
from 0% to 0.1%, typically from 0% to 0.05%, more typically from 0% to 0.02% of Spicy ingredients,
from 0% to 5%, typically from 0% to 4%, more typically from 0% to 3%, of Citrus ingredients, or any combination thereof;
wherein the total amount of Aromatic ingredients, Spicy ingredients, and Citrus ingredients is from 0% to 10%, typically from 0% to 5%, more typically from 0% to 3%, the amounts expressed by weight relative to the total weight of the composition.

In an embodiment, the mental performance enhancing composition comprises one or more Aromatic ingredients and one or more Spicy ingredients; one or more Spicy ingredients and one or more Citrus ingredients; one or more Aromatic ingredients and one or more Citrus ingredients; or one or more Aromatic ingredients, one or more Spicy ingredients, and one or more Citrus ingredients.

In an embodiment, the mental performance enhancing composition comprises from 0.1% to 5.5%, typically from 0.1% to 5%, more typically from 0.1% to 4.5%, of Aromatic ingredients, by weight relative to the total weight of the composition.

In an embodiment, the mental performance enhancing composition comprises from 0.1% to 3.5%, typically from 0.1% to 3%, more typically from 0.1% to 1% of Spicy ingredients, by weight relative to the total weight of the composition.

In an embodiment, the mental performance enhancing composition comprises from 0.1% to 5.5%, typically from 0.1% to 5%, more typically from 0.1% to 4.5%, of Citrus ingredients, by weight relative to the total weight of the composition.

In an embodiment, the total amount of Aromatic ingredients, Spicy ingredients, and Citrus ingredients is from 0.1% to 10%, typically from 0.1% to 6%, by weight relative to the total weight of the composition.

The Aromatic ingredients are selected from the group consisting of:
(+−)-1,7,7-trimethylbicyclo[2.2.1]heptan-2-one,
(−)-(5R)-5-isopropenyl-2-methyl-2-cyclohexen-1-one,
1,3,3-trimethyl-2-oxabicyclo[2.2.2]octane,
LAVANDIN GROSSO ARR,
5-methyl-2-(2-propanyl)cyclohexanone,
METHYL 2-HYDROXYBENZOATE,
thymol,
WORMWOOD OIL,
(+)-(1R,2R,4S)-1,3,3-trimethylbicyclo[2.2.1]heptan-2-ol,
4-methoxybenzaldehyde,
1-methoxy-4-[(1E)-1-propen-1-yl]benzene,
ANGELICA ROOT OIL,
METHYL 4-METHOXYBENZOATE,
ARTEMISIA OIL ARR,
BASIL OIL,
3-BUTYLIDENE-1-BENZO[C]FURANONE,
CAMOMILE,
ROMAN CHAMOMILE FLOWER OIL,
(+−)-2,2-DIMETHYL-3-METHYLENE-BICYCLO[2.2.1]HEPTANE,
alpha-PINENE,
CAMPHENE
EUCALYPTOL,
beta-PINENE,
SABINENE,
CARROTSEED OIL,
CARAWAY OIL,
(+)-(5S)-5-isopropenyl-2-methyl-2-cyclohexen-1-one,
CELERY SEED OIL,
(+−)-3-METHYL-5-PROPYL-2-CYCLOHEXEN-1-ONE,
1,4-EPOXY-P-MENTHANE,
1,8-EPOXY-P-MENTHANE,
CORIANDER OIL,
2,6-DIMETHYL-4-HEPTANOL,
1-METHOXY-4-PROPYLBENZENE,
(+−)-2,6-DIMETHYL-7-OCTEN-4-ONE,
1-methoxy-4-(2-propen-1-yl)benzene,
ESTRAGON OIL,
EUCALYPTUS BIOABSOLUTE,
FENNEL OIL BITTER,
FENUGREEK HYPERESSENCE,
(+−)-2-sec-butylcyclohexanone,
HYSSOP OIL,
EVERLASTING EO,
(−)-(1R,3R,4S)-8-P-MENTHEN-3-OL,
LAUREL LEAF OIL,
ASPIC NAT,
LAVENDER OIL,
LITSEA CUBEBA BERRY OIL,
MARJORAM EO,
PEPPERMINT OIL BRAZIL,
Peppermint USA Cascade OIL,
MENTHE POULIOT HE,
MINT SPICATA FW NATIVE,
(−)-L-MENTHOL,
(1 RS,3RS,4SR)-3-P-MENTHANOL,
MYRTLE OIL,
ORIGAN HE,
5-methyltricyclo[6.2.1.0~2,7~~]undecan-4-one,
REGLISSE ABS,
ROSEMARY EXTRACT,
2,2,8,10-tetramethylspiro[5.5]undec-8-en-1-one,
2,2,7,9-tetramethylspiro[5.5]undec-8-en-1-one,
DALMATIAN SAGE,
CLARY SAGE OIL,
4,5,6,7,8,9,10,11,12,13-DECAHYDROCYCLODODECA-1,3-OXAZOLE,
TAGETE EO,
2-methoxy-4-propylcyclohexanol,
(+−)-1-(3,3-dimethylcyclohexyl)ethenone,
(+−)-2,6,6-trimethylcycloheptanone,
(+−)-2-ethyl-4,4-dimethylcyclohexanone,
(1R,4S)-1,3,3-trimethylbicyclo[2.2.1]heptan-2-one,
1-isopropyl-4-methylbicyclo[3.1.0]hexan-3-one,
THYME RED EO,
1-(4-methoxyphenyl)-1-ethanone,
(+−)-1-pentyl-2-propenyl acetate,
4-methoxy-1-benzenemethanol,
STAR ANISE OIL,
BADIANE SFE EXTRACT,
5-isopropyl-2-methylphenol,
CEDARLEAF OIL,
5-methyl-3-heptanone,
(+−)-3-(4-methoxyphenyl)-2-methylpropanal,
WINTERGREEN OIL NAT,
2-butyl-4,4,6-trimethyl-1,3-dioxane,
methyl 2-hydroxybenzoate,
ethyl 2-hydroxybenzoate,
(−)-propyl (2S)-2-[(2-methyl-2-butanyl)oxy]propanoate,
SPEARMINT OIL ORG,
and any combination thereof.

The Spicy ingredients are selected from the group consisting of:
(E)-3-PHENYL-2-PROPENAL,
(−)-(1R,4E,9S)-4,11,11-trimethyl-8-methylenebicyclo[7.2.0]undec-4-ene,
2-methoxy-4-(2-propen-1-yl)phenol,
2-methoxy-4-[(1E)-1-propen-1-yl]phenol,
1,2-dimethoxy-4-[1-propen-1-yl]benzene,
4-(2-propanyl)benzaldehyde,
4-allyl-2-methoxyphenyl acetate,
(2E)-2-methyl-3-phenyl-2-propenal,
CASSIA OIL,
CINNAMON OIL CEYLON,
CARDAMOM OIL,
(E)-3-PHENYL-2-PROPENENITRILE,
2-methoxy-4-propylphenol,
ELEMI OIL,
4-ETHYL-2-METHOXYPHENOL,
GINGER,
ENGLISH CLOVE OIL,
GIROFLE TERPENES,
NUTMEG ABSOLUTE PURE AND NAT,
PIMENTA BERRY OIL,
BLACK PEPPER STEAM DIST.,
SICHUAN PEPPER (XANTHOXYLUM/ZANTHOXYLUM PIPERITUM),
(+−)-2,3,3-TRIMETHYL-1-INDANONE,
SAFRAN FIRBEST,
2,6,6-TRIMETHYL-1,3-CYCLOHEXADIENE-1-CARBALDEHYDE,
2-methoxy-4-vinylphenol,
4-isopropyl-1-benzenemethanol,
(1 RS,2SR,5RS,8SR)-4,4,8-trimethyltricyclo[6.3.1.0~2,5-]dodec-1-yl formate,
(1RS,2RS,5RS,8SR)-4,4,8-trimethyltricyclo[6.3.1.0~1,5-]dodec-2-yl formate,
CUMIN,
2-ethoxy-4-(methoxymethyl)phenol,
2,6,6-trimethyl-2-cyclohexene-1,4-dione,
turmeric oil,
and any combination thereof.

The Citrus ingredients are selected from the group consisting of:
(+−)-1,5-DIMETHYL-1-VINYL-4-HEXENYL ACETATE,
(+−)-2-(4-methyl-3-cyclohexen-1-yl)-2-propanyl acetate,
(−)-(R)-3,7-DIMETHYL-6-OCTENENITRILE,
trimethyl-4-phenyl-1,3-dioxane,
3,7-dimethyl-2,6-nonadienenitrile,
3,7-dimethyl-3,6-nonadienenitrile,
(+)-LIMONENE,
(4Z)-4-dodecenal,
(+)-(R)-3,7-DIMETHYL-6-OCTENAL,
3,7-DIMETHYL-2,6-OCTADIENAL,
ORANGE TERP CITRONOVA,
cis-4-isopropyl-1-methylcyclohexyl acetate,
2-(4-methylcyclohexyl)-2-propanyl acetate,
trans-4-isopropyl-1-methylcyclohexyl acetate,
2-(6,6-DIMETHYL-BICYCLO[3.1.1]HEPT-2-EN-2-YL)ETHYL ACETATE,
(+−)-3,7-dimethyl-1,6-nonadien-3-yl acetate,
1-METHYL-1,3-CYCLOHEXADIENE,
2-METHYL-1,3-CYCLOHEXADIENE,
3-METHYLENE-1-CYCLOHEXENE,
BERGAMOT FUROCOUMARIN-FREE,
BERGAMOTE ESS,
CEDRAT ESSENTIAL OIL,
LEMON OIL CALIF NAEFCO BRAND,
3-methyl-5-phenyl-2-pentenenitrile,
(+−)-2,6-DIMETHYL-7-OCTEN-2-OL,
4-(2-methoxy-2-propanyl)-1-methylcyclohexene,
GRAPEFRUIT ARR DECOL,
KAFFIR LIME LEAF OIL,
LIME OIL DIST,
LIME EXPR SSQT FAB,
LIME OIL TERPENES,
1,4/8-EPOXY-P-MENTHANE,
LIME OXIDE,
MANDARINE SFUMA,
6,6-DIMETHOXY-2,5,5-TRIMETHYL-2-HEXENE,
3,6,7-TRIMETHYL-2,6-OCTADIENAL,
(+)-(4R,4aS,6R)-4,4a-dimethyl-6-(1-propen-2-yl)-4,4a,5,6,7,8-hexahydro-2(3H)-naphthalenone,
ORANGE ESSENCE,
ORANGE COLORLESS ESSENTIAL OIL MOLECULAR DISTILLATION,
ORANGE TERP CITRONOVA,
PORTUGAL FLORIDE DECOL,
DECANAL,
DODECANAL,
beta-SINENSAL,
GERANIAL,
NERAL,
alpha-SINENSAL,
NOOTKATONE,
ELEMOL,
1,5,9-trimethyl-4,8-decadienyl acetate,
octyl acetate,
1-decanol,
(+−)-2-methyl-3-(4-methylphenyl)propanal,
(+)-(1 S,6R)-3,7,7-trimethylbicyclo[4.1.0]hept-3-ene,
(2Z)-1,1-dimethoxy-3,7-dimethyl-2,6-octadiene,
(2E)-1,1-dimethoxy-3,7-dimethyl-2,6-octadiene,
(2Z)-1,1-diethoxy-3,7-dimethyl-2,6-octadiene,
(2E)-1,1-diethoxy-3,7-dimethyl-2,6-octadiene,
LEMON CP FCR DECOL,
LEMON OIL DLE,
LEMON ESS 1341 ARR,
LEMON OIL WINTER TORCHIO DECOL,
CITRON SFUMA DECOL,
CITRON SFUMA,
Lemon oil Primofiore,
TETRAROME CITRON,
CITRONNELLE ESS BIO
CITRONELLA OIL,
CITRONELLA CEYLON EO,
methyl 2-(methylamino)benzoate,
2,6-dimethyl-7-octen-2-ol,
1,1,5-trimethyl-6-heptenyl formate,
alpha-terpinene,
(+−)-limonene,
alpha-terpinolene,
EUCALYPTUS CITRIODORA OIL,
EUCALYPTUS CITRIODORA ORGANIC EO,
LEMONGRASS,
(+−)-2-ethenyl-2,6,6-trimethyltetrahydro-2H-pyran,
(+)-(3S)-3-[(1R)-4-methyl-3-cyclohexen-1-yl]butanal,
(+)-(3R)-3-[(1R)-4-methyl-3-cyclohexen-1-yl]butanal,
LITSEA CUBEBA BERRY OIL,
(3Z)-3,12-tridecadienenitrile,
(3E)-3,12-tridecadienenitrile,
methyl 2-(methylamino)benzoate,
methyl octanoate,
ORANGE SANGUINE DM,
(+−)-3-methyl-5-phenylpentanal,
MELALEUCA,
(+−)-2,6-dimethyl-2-octanol,
(4E)-4-decenal,
(+−)-3,7-dimethyloctanal,
and any combination thereof.

In another embodiment, the total amount of Aromatic ingredients selected from the group consisting of:
(+−)-1,7,7-trimethylbicyclo[2.2.1]heptan-2-one,
(−)-(5R)-5-isopropenyl-2-methyl-2-cyclohexen-1-one,
1,3,3-trimethyl-2-oxabicyclo[2.2.2]octane,
LAVANDIN GROSSO ARR,
5-methyl-2-(2-propanyl)cyclohexanone,
METHYL 2-HYDROXYBENZOATE,
thymol,
and any combination thereof;

Spicy ingredients selected from the group consisting of:
(E)-3-PHENYL-2-PROPENAL,
(−)-(1R,4E,9S)-4,11,11-trimethyl-8-methylenebicyclo[7.2.0]undec-4-ene,
2-methoxy-4-(2-propen-1-yl)phenol,
2-methoxy-4-[(1E)-1-propen-1-yl]phenol,
1,2-dimethoxy-4-[1-propen-1-yl]benzene,
4-(2-propanyl)benzaldehyde, and any combination thereof;
and Citrus ingredients selected from the group consisting of:
(+−)-1,5-DIMETHYL-1-VINYL-4-HEXENYL ACETATE,
(+−)-2-(4-methyl-3-cyclohexen-1-yl)-2-propanyl acetate,
(−)-(R)-3,7-DIMETHYL-6-OCTENENITRILE,
trimethyl-4-phenyl-1,3-dioxane,
3,7-dimethyl-2,6-nonadienenitrile,
3,7-dimethyl-3,6-nonadienenitrile,
(+)-LIMONENE,
(4Z)-4-dodecenal,
(+)-(R)-3,7-DIMETHYL-6-OCTENAL,
3,7-DIMETHYL-2,6-OCTADIENAL,
ORANGE TERP CITRONOVA, and any combination thereof;
is less than 3%, typically less than 2.5%, more typically less than 2%, by weight relative to the total weight of the composition.

The mental performance enhancing compositions described herein may further comprise additional ingredients typically used in the perfumery industry, such as a perfumery carrier.

As used herein, “perfumery carrier” refers to a material which is practically neutral from a perfumery point of view, i.e., that it does not significantly alter the organoleptic properties of perfuming ingredients. Said carrier may be a liquid or a solid. Exemplary liquid carriers include, but are not limited to, an emulsifying system, i.e., a solvent and a surfactant system, or a solvent typically used in perfumery. A detailed description of the nature and type of solvents commonly used in perfumery cannot be exhaustive. However, suitable solvents include, but are not limited to, glycols, such as propylene or butylene glycol; glycerol; dipropyleneglycol and its monoether, 1,2,3-propanetriyl triacetate, dimethyl glutarate, dimethyl adipate, 1,3-diacetyloxypropan-2-yl acetate, diethyl phthalate, isopropyl myristate, rosin resins (such as Abalyn® available from Eastman), benzyl benzoate, benzyl alcohol, 2-(2-ethoxyethoxy)-1-ethanol, tri-ethyl citrate, and mixtures thereof; and naturally derived solvents, such as glycerol and vegetable oils, including palm oil, sunflower oil, linseed oil, and mixtures thereof. In some instances, for example, the perfumery carrier may be ethanol, water/ethanol mixtures, limonene or other terpenes, isoparaffins such as those known under the trademark Isopar® (available from Exxon Chemical) or glycol ethers and glycol ether esters, such as those known under the trademark Dowanol® (available from Dow Chemical Company), or hydrogenated castors oils such as those known under the trademark Cremophor® RH 40 (available from BASF).

Solid carriers are materials to which the mental performance enhancing composition or some element of the mental performance enhancing composition can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. Suitable solid carriers include, but are not limited to, absorbing gums or polymers or inorganic materials, such as porous polymers, cyclodextrines, dextrines, maltodextrines wood-based materials, organic or inorganic gels, clays, gypsum talc or zeolites.

Other suitable solid carriers include encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof.

Other suitable encapsulating materials are described in reference texts known to those of skill in the art, such as H. Scherz, Hydrokolloide: Stabilisatoren, Dickungs-und Geliermittel in Lebensmitteln, Band 2 der Schriftenreihe Lebensmittelchemie, Lebensmittelqualitst, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

Other exemplary solid carriers include core-shell capsules with resins of aminoplast, polyamide, polyester, polyurea or polyurethane type, and mixtures threof, made using techniques well-known to those of ordinary skill in the art, such as phase separation induced by polymerization, interfacial polymerization, coacervation, or a combination thereof, optionally in the presence of a polymeric stabilizer or of a cationic copolymer.

Resins may be produced by the polycondensation of an aldehyde (e.g., formaldehyde, 2,2-dimethoxyethanal, glyoxal, glyoxylic acid or glycolaldehyde, and mixtures thereof) with an amine such as urea, benzoguanamine, glycoluryl, melamine, methylol melamine, methylated methylol melamine, guanazole and the like, as well as mixtures thereof. Alternatively, one may use preformed resins like alkylolated polyamines such as those commercially available under the trademark Urac® (origin: Cytec Technology Corp.), Cymel® (origin: Cytec Technology Corp.), Urecoll® or Luracoll® (origin: BASF).

Other suitable resins are the those produced by the polycondensation of a polyol, like glycerol, and a polyisocyanate, for example, a trimer of hexamethylene diisocyanate, a trimer of isophorone diisocyanate or xylylene diisocyanate or a Biuret of hexamethylene diisocyanate or a trimer of xylylene diisocyanate with trimethylolpropane (marketed as Takenate® by Mitsui Chemicals), among which a trimer of xylylene diisocyanate with trimethyloIpropane and a Biuret of hexamethylene diisocyanate are of mention.

The encapsulation of perfumes by polycondensation of amino resins, namely melamine-based resins with aldehydes is well-known in the art. Pertinent publications include, but are not limited to, K. Dietrich et al. Acta Polymerica, 1989, vol. 40, pages 243, 325 and 683, as well as 1990, vol. 41, page 91 and U.S. Pat. No. 4,396,670 issued Aug. 2, 1983. The general knowledge in encapsulation technology is very significant and cannot be exhaustive. More recent publications of pertinence, which disclose suitable uses of such microcapsules, are represented, for example, by the article of K. Bruyninckx and M. Dusselier, ACS Sustainable Chemistry & Engineering, 2019, vol. 7, pages 8041-8054. These publications are incorporated herein by reference.

The mental performance enhancing composition may optionally comprise at least one perfumery adjuvant.

The at least one perfumery adjuvant is an ingredient capable of imparting an additional added benefit such as a color, a particular light resistance, chemical stability, etc. A detailed description of the nature and type of adjuvant commonly used in perfuming compositions cannot be exhaustive, but it has to be mentioned that said ingredients are well known to a person skilled in the art.

Exemplary perfumery adjuvants include, but are not limited to, viscosity agents (e.g., surfactants, thickeners, gelling and/or rheology modifiers), stabilizing agents (e.g., preservatives, antioxidant, heat/light and or buffers or chelating agents, such as BHT), coloring agents (e.g., dyes and/or pigments), preservatives (e.g. antibacterial or antimicrobial or antifungal or anti irritant agents), abrasives, skin cooling agents, fixatives, insect repellants, ointments, vitamins and mixtures thereof.

Fixatives, also known as “modulators”, are agents having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived. Suitable modulators include, but are not limited to, methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and any combination thereof.

The mental performance enhancing compositions described herein may further comprise one or more other odorant ingredients so long as the presence and/or concentrations of the said odorant ingredient or ingredients do not alter or otherwise disrupt the ability of the composition to enhance mental performance in a subject. Such odorant ingredients include, but are not limited to, (+−)-1-(3-CYCLOOCTEN-1-YL)-1-PROPANOL, ETHYL CYCLOHEXANECARBOXYLATE, 7-(3-METHYLBUTYL)-2H-1,5-BENZODIOXEPIN-3(4H)-ONE, METHYL (E)-4,7-OCTADIENOATE, 11-OXA-16-HEXADECANOLIDE, (2E,5Z)-5,6,7-TRIMETHYL-2,5-OCTADIEN-4-ONE, 3-METHYL-5-CYCLOTETRADECEN-1-ONE, (+−)-2-TERT-BUTYL-5-METHYL-2-PROPYL-2,5-DIHYDROFURAN, (+−)-(Z)-1-(3-CYCLOOCTEN-1-YL)ETHANONE, 2-ETHYL-N-METHYL-N-(3-METHYLPHENYL)BUTANAMIDE, (3Z)-1-METHYL-3-HEXENYL SALICYLATE, (+−)-6-METHOXY-2,6-DIMETHYLOCTANAL, cyclohexylidene(2-methylphenyl)acetonitrile, (+−)-2-(2,2,7,7-tetramethyltricyclo[6.2.1.0~1,6~]undec-5-en-5-yl)-1-propanol, (+−)-4-vinyl-1-cyclohexene-1-carbaldehyde, (+−)-2-[(3,5-dimethyl-3-hexen-2-yl)oxy]-2-methylpropyl cyclopropanecarboxylate, (+−)-1-methoxy-7,9-dimethyl-2-oxaspiro[5.5]undec-8-ene, (+−)-1-ethoxy-7,9-dimethyl-2-oxaspiro[5.5]undec-8-ene, (3S,5R,8S)-5-isopropenyl-3,8-dimethyl-1,2,3,4,5,6,7,8-octahydro-1-azulenol, (+−)-8-(2-butanyl)-5,6,7,8-tetrahydroquinoline, (4Z)-9-hydroxy-5,9-dimethyl-4-decenal, (+−)-2-[(1E)-3-ethoxy-1-buten-1-yl]-1,3,3-trimethylcyclohexene, 3-(4-isobutyl-2-methylphenyl)propanal, (+−)-[ 1-methyl-2-(5-methyl-4-hexen-2-yl)cyclopropyl]methanol, (+−)-2,4,7-trimethyl-6-octen-1-ol, METHYL 2-METHYLBENZOATE, (+−)-METHYL 3-METHYL-1-CYCLOHEXANECARBOXYLATE, (+−)-1-(2,4-DIMETHYL-3-CYCLOHEXEN-1-YL)-1-PROPANOL, oxacycloheptadecan-2-one, ((1 RS,2RS)-2,4-dimethylcyclohex-3-enyl)methyl acetate, (+)-(1 S)-1-[(1R)-3,3-dimethylcyclohexyl]ethyl ethyl propanedioate, (+)-(1 S)-1-[(1R)-3,3-dimethylcyclohexyl]ethyl ethyl propanedioate, (+−)-4-METHYL-2-PROPYL-1,3-OXATHIANE, ETHYL 3-(1-CYCLOHEXEN-1-YL)PROPANOATE, (+)-(1R,4S,9S,10R,13S)-5,5,9,13-tetramethyl-14,16-dioxatetracyclo[11.2.1.0~1,10-.0-4,9-]hexadecane, 2,5-DIMETHYL-5-PHENYL-1-HEXEN-3-ONE, (3Z)-3-HEXENYL CYCLOPROPANECARBOXYLATE, (Z)-6-DECENAL, (1 RS,2SR,6SR,7RS,8RS)-tricyclo[5.2.1.0(2,6)]dec-8-ylacetaldehyde, 3Aa-ETHYL-PERHYDRO-6,6,9Aa-TRIMETHYL-5AbH,9BaH-NAPHTHO[2,1-B]FURAN, 3-(4-ISOPROPYL-1-PHENYL)PROPANAL, (+−)-3-ETHYL-PERHYDRO-BENZO[B]FURAN-2-ONE, 5-METHYL-5-PHENYL-3-HEXANONE, (+−)-CIS-2-ETHYL-4-METHYL-1,3-OXATHIANE, (+−)-1-ETHYL-2-(3-METHYLBUTYL)-1-CYCLOPENTANOL, 3-(trans-4-isobutylcyclohexyl)propanal, METHYL 2,4-DIHYDROXY-3-METHYLBENZOATE, 1,1-DIMETHYL-2-PHENYLETHYL ISOBUTYRATE, (+−)-2-methoxy-4-(4-methyl-3,6-dihydro-2H-pyran-2-yl)phenol, (+−)-2,2,6,6,7,8,8-heptamethyl-4,5,6,7,8,8b-hexahydro-3aH-indeno[4,5-d][1,3]dioxole, (+−)-4-(HEPTYLOXY)-3-METHYLBUTANAL, (3E)-4-methyl-3-decen-5-one, (+−)-2-(5-isopropyl-2-methyltetrahydrothiophen-2-yl)ethanol, (+−)-ethyl 4-methyl-2-oxocyclohexanecarboxylate, (1 RS,2RS,4RS)-ethyl bicyclo[2.2.1]hept-5-ene-2-carboxylate, (+−)-4,6-dimethyl-2-(1-phenylethyl)-3,6-dihydro-2H-pyran, 2-methyl-1-(5,5,6-trimethylbicyclo[2.2.1]hept-2-yl)-2-propanol, tricyclo[5.2.1.0~2,6-]dec-3-en-8-yl butyrate, (+−)-7,7,8,9,9-pentamethyl-6,7,8,9-tetrahydro-5H-cyclopenta[h]quinazoline, (6aRS,9aRS)-7,7,8,9,9-pentamethyl-6,6a,7,8,9,9a-hexahydro-5H-cyclopenta[h]quinazoline, 5,7,8-trimethyltricyclo[5.2.1.0~2,6-]decan-8-ol, (+−)-2-methyl-3-pentanyl (2E)-2-butenoate, 3-methyl-1-benzofuran-5-ol, (−)-4,6,6,7,8,8-hexamethyl-1,3,4,6,7,8-hexahydrocyclopenta[g]isochromene, (+−)-3a-isopropyl-2,6-dimethyloctahydro-1-benzofuran, 5-(hexyloxy)-2,2-dimethyltetrahydrofuran, (+−)-4-ethylidene-2-propoxycyclohexanol, 3-[(1R,5S)-6,6-dimethylbicyclo[3.1.1]hept-2-en-2-yl]-2,2-dimethylpropanenitrile, (+−)-(1E)-1-(4,6-dimethyl-3-cyclohexen-1-yl)-2-methyl-1-penten-3-one, (+−)-(3E)-3-(methoxymethylene)tricyclo[5.2.1.0~2,6-]decane, (2E,5E)-2-methoxy-6-methyl-2,5-octadiene, (14S)-14-methyloxacyclohexadec-10-en- 2-one, 5-propyltetrahydro-2H-pyran-2-one, (2Z)-(2,4,4-trimethylcyclopentylidene)acetonitrile, 6-cyclopentylidenehexanal, (4Z)-6-(2,2,3-trimethyl-3-cyclopenten-1-yl)-4-hexenal, (+−)-(2Z)-3,5,7-trimethyl-2-octenenitrile, 3,5,7-trimethyloctanenitrile, 6-(2,4,4-trimethylcyclopentylidene)hexanal, (1SR,6RS,8SR)-2,2-dimethyl-8-propyltricyclo[6.2.1.0~1,6-]undecan-7-one, 2,6-dimethyl-4-(2,2,3-trimethylcyclopent-3-en-1-yl)cyclohex-2-en-1-ol, 5-(6-hepten-1-yl)dihydro-2(3H)-furanone, (+−)-(1Z,5E)-9-methoxy-2,5,10-trimethyl-1,5-cyclododecadiene, 2-(3,3-dimethyl-1-cyclohexen-1-yl)-2,5,5-trimethyl-1,3-dioxane, 8-CYCLOHEXADECEN-1-ONE, 4-DECYLPYRIDINE, (+−)-8EXO-ETHOXY-EXO-TRICYCLO[5.2.1.0(2,6)]DECANE, (+−)-ethyl tetrahydro-2-furancarboxylate, 3-METHYL-4-DECANOLIDE, (Z)-4-ETHYL-3-OCTENENITRILE, (+)-3-[(4R)-4-isopropyl-1-cyclohexen-1-yl]propanal, bicyclo[2.2.1]hept-5-en-2-yl acetate, (+−)-ethyl 2-cyclopenten-1-ylacetate, (+−)-2-METHYLDODECANAL, 4-(3-METHYLBUTYL)CYCLOHEXANOL, 6-methyl-4-(2,2,3-trimethyl-3-cyclopenten-1-yl)-2-cyclohexen-1-ol, 2-((1RS,2SR)-2-ethylcyclohexyloxy)acetaldehyde, (+−)-5-cyclohexyl-2-methyl-1-pentanol, [(cis-4-methylcyclohexyl)oxy]acetaldehyde, {[cis-4-(2-propanyl)cyclohexyl]oxy}acetaldehyde, {[trans-4-(2-methyl-2-propanyl)cyclohexyl]oxy}acetaldehyde, (1R,3S,5R,7R,8R,10S,13R)-5,7,9,9,13-pentamethyl-5-propyl-4,6-dioxatetracyclo[6.5.1.0~1,10-.0-3,7-]tetradecane, (+−)-2-methyl-5-propyl-2-cyclohexen-1-one, 3,4-dimethylbenzonitrile3,4-dimethylbenzonitrile, diethyl (2E)-2-methyl-2-butenedioate, 5-MERCAPTO-5-METHYL-3-HEXANONE, (+−)-3-(4-ISOBUTYLPHENYL)-2-METHYLPROPANAL, (+−)-2,5,5-trimethyl-1,2,3,4,4a,5,6,7-octahydro-2-naphthalenol, (+−)-ethyl 3-mercapto-2-methylbutanoate, (−)-(3S,6E)-3,7,11-trimethyl-6,10-dodecadienal, (+)-(1 S,2R)-1,2,3,3-TETRAMETHYL-BICYCLO[2.2.1]HEPTAN-2-OL (1S,4R)-1,2,3,3-TETRAMETHYL-BICYCLO[2.2.1]HEPTAN-2-OL, 3-methyl-5-[(1R)-2,2,3-trimethylcyclopentyl]-2-pentanone, 2-ethoxy-4-(hydroxymethyl)phenol, (+−)-3-cyclohexen-1-ylmethyl 2-hydroxypropanoate, 2-methoxy-2-methyl-heptane, allyl (1R,6S)-2,2,6-trimethylcyclohexanecarboxylate, (+−)-6-hydroxy-2,6-dimethylheptanal, 3-methyl-2-pentyl-2-cyclopenten-1-one, 2,2-dimethyl-3-(3-methylphenyl)-1-propanol, BELLANONE, CANTALOOP, CEYLANIL, CYCLAMENTIOL, DIVINIRIS, EDENFLOR, FLOWERPOOL, and the like.

The mental performance enhancing compositions according to the present disclosure may be prepared according to any method known to those of ordinary skill in the art. The ordinarily-skilled artisan is perfectly able to design optimal formulations for the desired effect by admixing the above mentioned components to arrive at the desired composition by applying standard knowledge and concepts known to those of ordinary skill and by utilizing routine optimization methodologies.

Exemplary compositions include, but are not limited to:

### Fragrance A

| | Ingredient | Amount |
|---|---|---|
| Powdery (wt %) | 2-chromenone | 2.06 |
| Musky (wt %) | oxacyclohexadecan-2-one | 20.60% |
| | (9Z)-9-cycloheptadecen-1-one | 0.02% |
| Floral (wt %) | (+−)-3,7-dimethyl-6-octen-1-ol | 2.10% |
| | (+−)-(3E)-3-methyl-4-(2,6,6- | 1.00% |
| | trimethyl-2-cyclohexen-1-yl)-3-buten- | |
| | 2-one (A) + (+−)-(1E)-1- | |
| | (2,6,6-trimethyl-2-cyclohexen-1-yl)- | |
| | 1-penten-3-one (B | |
| | (−)-(2S)-1-oxo-1-(2-propanyloxy)- | 1.00% |
| | 2-propanyl | |
| | 2,2-dimethylpropanoate | |
| Total enhancing | | 26.8 |
| ingredients | | |
| Spicy (wt %) | 2-methoxy-4-(2-propen-1-yl)phenol | 2.1 |
| Total disrupting | | 2.1 |
| ingredients | | |

### Fragrance G

| | Ingredient | Amount |
|---|---|---|
| Floral (wt %) | methyl 2-((1RS,2RS)-3-oxo-2- | 45.20% |
| | pentylcyclopentyl)acetate | |
| | benzyl acetate | 12.50% |
| | (2E)-2-benzylideneoctanal | 10.00% |
| | (+−)-3,7-dimethyl-1,6-octadien-3-ol | 10.00% |
| | 2-phenylethanol | 5.00% |
| | benzyl salicylate | 4.00% |
| | benzyl propanoate | 3.00% |
| | (+−)-(Z)-7-decen-4-olide | 1.00% |
| | methyl benzoate | 0.20% |
| | methyl {(1RS,2RS)-3-oxo-2-[(2Z)-2-penten- | 0.20% |
| | 1-yl]cyclopentyl}acetate | |
| | methyl cyclopentylideneacetate | 0.10% |
| | (1RS,2SR)-2-pentylcyclopentanol (A) + | 0.10% |
| | (1RS,2RS)-2-pentylcyclopentanol (B) | |
| | (+)-methyl (1R)-cis-3-oxo-2-pentyl-1- | 0.10% |
| | cyclopentaneacetate | |
| | (+−)-2-pentylcyclopentanone | 0.10% |
| | JASMIN INDES ABS SUPER | 0.01% |
| Total enhancing | | 91.7 |
| ingredients | | |
| Aromatic (wt %) | ethyl 2-hydroxybenzoate | 0.3 |
| Spicy (wt %) | 4-allyl-2-methoxyphenyl acetate | 0.50% |
| | 2-methoxy-4-(2-propen-1-yl)phenol | 0.20% |
| Total disrupting | | 1.0 |
| ingredients | | |

### Fragrance H

| | Ingredient | Amount |
|---|---|---|
| Powdery (wt %) | 4-hydroxy-3-methoxybenzaldehyde | 15.60% |
| | 3-ethoxy-4-hydroxybenzaldehyde | 9.40% |
| Musky (wt %) | 1,4-dioxacycloheptadecane-5,17-dione | 12.50% |
| | oxacyclohexadecan-2-one | 3.10% |
| Floral (wt %) | methyl 2-((1RS,2RS)-3-oxo-2- | 50.00% |
| | pentylcyclopentyl)acetate | |
| | 4-(2,6,6-trimethyl-1-cyclohexen-1-yl)-2- | 2.50% |
| | butanone | |
| Total enhancing | | 93.1 |
| ingredients | | |

### Fragrance I

| | Ingredient | Amount |
|---|---|---|
| Powdery (wt %) | 2-chromenone | 0.30% |
| | 4-(4-hydroxyphenyl)-2-butanone | 0.01% |
| Musky (wt %) | 2-{(1RS)-1-[(1SR)-3,3- | 33.00% |
| | dimethylcyclohexyl]ethoxy}-2-oxoethyl | |
| | propionate (A) + 2-{(1RS)-1-[(1RS)-3,3- | |
| | dimethylcyclohexyl]ethoxy}-2-oxoethyl | |
| | propionate (B) + 2-oxo-2-{[(1RS,2RS)-2,6,6- | |
| | trimethylcycloheptyl]oxy}ethyl propionate | |
| | (C) + 2-oxo-2-{[(1RS,2SR)-2,6,6- | |
| | trimethylcycloheptyl]oxy}ethyl propionate (D) | |
| | oxacyclohexadecan-2-one | 22.00% |
| | 1,4-dioxacycloheptadecane-5,17-dione | 16.40% |
| | 1-OXA-12-CYCLOHEXADECEN-2-ONE (A) + | 7.20% |
| | 1-OXA-13-CYCLOHEXADECEN-2-ONE (B) | |
| | (1S)-1-[(1R)-3,3-dimethylcyclohexyl]ethyl | 5.50% |
| | [(2R,3R)-3-ethyl-2-oxiranyl]acetate (A) + | |
| | (1S)-1-[(1R)-3,3-dimethylcyclohexyl]ethyl | |
| | [(2S,3S)-3-ethyl-2-oxiranyl]acetate (B) | |
| Floral (wt %) | methyl 2-((1RS,2RS)-3-oxo-2- | 8.70% |
| | pentylcyclopentyl)acetate | |
| | (+−)-3,7-dimethyl-1,6-octadien-3-ol | 1.10% |
| | 2-phenylethanol | 1.10% |
| | (+−)-(E)-3-methyl-4-(2,6,6-trimethyl-2- | 0.50% |
| | cyclohexen-1-yl)-3-buten-2-one | |
| | (+−)-3,7-dimethyl-6-octen-1-ol | 0.50% |
| | benzyl acetate | 0.30% |
| | (+−)-7-hydroxy-3,7-dimethyloctanal | 0.30% |
| | 3,7,11-trimethyl-1,6,10-dodecatrien-3-ol | 0.20% |
| | (+−)-(E)-3-methyl-4-(2,6,6-trimethyl-2- | 0.20% |
| | cyclohexen-1-yl)-3-buten-2-one (A) + (E)-1- | |
| | (2,6,6-trimethyl-2-cyclohexen-1-yl)-1-penten- | |
| | 3-one (B) + (+−)-(E)-1-(2,2-dimethyl-6- | |
| | methylene-1-cyclohexyl)-1-penten-3-one (C) + | |
| | (E)-1-(2,6,6-trimethyl-1-cyclohexen-1-yl)-1- | |
| | penten-3-one (D) | |
| | (+)-methyl (1R)-cis-3-oxo-2-pentyl-1- | 0.10% |
| | cyclopentaneacetate | |
| | (+−)-2,4-dimethyl-4,4a,5,9b- | 0.10% |
| | tetrahydroindeno[1,2-d][1,3]dioxine (ISOMER | |
| | A) + (+−)-2,4-dimethyl-4,4a,5,9b- | |
| | tetrahydroindeno[1,2-d][1,3]dioxine (ISOMER | |
| | B) (A + B) | |
| | YLANG EO EXTRA | 0.10% |
| | benzyl salicylate | 0.10% |
| | JASMIN INDES ABS SUPER | 0.02% |
| | TUBEREUSE INFUSION | 0.01% |
| | benzyl acetate | 0.004% |
| | (+−)-(2,5-dimethyl-2,3-dihydro-1H-inden-2- | 0.002% |
| | yl)methanol | |
| | benzyl propanoate | 0.001% |
| | 2-phenylethyl acetate | 0.0003% |
| | (5Z)-6,10-dimethyl-5,9-undecadien-2-one (A) + | 0.0002% |
| | (5E)-6,10-dimethyl-5,9-undecadien-2-one (B) | |
| | methyl benzoate | 0.0002% |
| | (+−)-(3E)-3-methyl-4-(2,6,6-trimethyl-2- | 0.0002% |
| | cyclohexen-1-yl)-3-buten-2-one (A) + (+−)- | |
| | (1E)-1-(2,6,6-trimethyl-2-cyclohexen-1-yl)-1- | |
| | penten-3-one (B) | |
| | 3-methyl-2-[(2Z)-2-penten-1-yl]-2- | 0.0001% |
| | cyclopenten-1-one | |
| | (+−)-6-[(2Z)-2-penten-1-yl]tetrahydro-2H- | 0.0001% |
| | pyran-2-one | |
| | 1-nonanol | 0.000001% |
| | (2RS,4SR)-4-methyl-2-(2-methyl-1-propen-1- | 0.0000004 |
| | yl)tetrahydro-2H-pyran (A) + (2RS,4RS)-4- | |
| | methyl-2-(2-methyl-1-propen-1-yl)tetrahydro- | |
| | 2H-pyran (B) | |
| | (3E)-4-(2,6,6-trimethyl-1-cyclohexen-1-yl)-3- | 0.0000003 |
| | buten-2-one | |
| | (+−)-3-(4-isopropylphenyl)-2-methylpropanal | 0.0000002 |
| | (+−)-(3E)-4-(2,6,6-trimethyl-2-cyclohexen-1- | 0.0000002 |
| | yl)-3-buten-2-one (A) + (3E)-4-(2,6,6- | |
| | trimethyl-1-cyclohexen-1-yl)-3-buten-2-one | |
| | (B); | |
| Total enhancing | | 97.9 |
| ingredients | | |
| Aromatic (wt %) | 1-methoxy-4-propylbenzene | 0.11 |
| | methyl 2-hydroxybenzoate | 0.0002 |
| | CARROT SEED OIL | 0.00001 |
| | IMMORTELLE ABSOLUTE | 0.000004 |
| Citrus (wt %) | (2Z)-3,7-dimethyl-2,6-octadienal (A) + (2E)- | 0.0000002 |
| | 3,7-dimethyl-2,6-octadienal (B) | |
| Spicy (wt %) | 2-methoxy-4-(2-propen-1-yl)phenol | 0.001 |
| | (E)-2-methoxy-4-(1-propenyl)phenyl acetate | 0.0003 |
| | 2-methoxy-4-[(1E)-1-propen-1-yl]phenol | 0.0003 |
| | 2-methoxy-4-vinylphenol | 0.00000015 |
| Total disrupting | | 0.11 |
| ingredients | | |

### Fragrance J

| | Ingredient | Amount |
|---|---|---|
| Powdery (wt %) | 2-chromenone | 4.62% |
| | 3-ethoxy-4-hydroxybenzaldehyde | 0.35% |
| | (3S,3aS,6R,7aR)-3,6-dimethylhexahydro-1- | 0.01% |
| | benzofuran-2(3H)-one | |
| Musky (wt %) | oxacyclohexadecan-2-one | 5.78% |
| Floral (wt %) | benzyl salicylate | 6.94% |
| | benzo[d][1,3]dioxole-5-carbaldehyde | 2.89% |
| | (+−)-3,7-dimethyl-1,6-octadien-3-ol | 2.31% |
| | methyl benzoate | 0.01% |
| Total | | 22.9 |
| enhancing | | |
| ingredients | | |
| Aromatic (wt %) | 4-methoxybenzaldehyde | 1.16 |
| Spicy (wt %) | (−)-(1R,4E,9S)-4,11,11-trimethyl-8- | 0.34 |
| | methylenebicyclo[7.2.0]undec-4-ene | |
| Total | | 1.5 |
| disrupting | | |
| ingredients | | |

### Fragrance K

| | Ingredient | Amount |
|---|---|---|
| Powdery (wt %) | 4-hydroxy-3-methoxybenzaldehyde | 15.60% |
| | 3-ethoxy-4-hydroxybenzaldehyde | 9.40% |
| | VANILLE TAHITENSIS SFE 25 TEC | 0.20% |
| Musky (wt %) | 1,4-dioxacycloheptadecane-5,17-dione | 12.50% |
| | oxacyclohexadecan-2-one | 3.10% |
| Floral (wt %) | methyl 2-((1RS,2RS)-3-oxo-2- | 49.90% |
| | pentylcyclopentyl)acetate | |
| | 4-(2,6,6-trimethyl-1-cyclohexen-1-yl)-2- | 2.50% |
| | butanone | |
| Total | | 93.10 |
| enhancing | | |
| ingredients | | |

In the second aspect, the present disclosure relates to a mental performance enhancing composition comprising:
one or more Musky ingredients and one or more Powdery ingredients;
one or more Powdery ingredients and one or more Floral ingredients;
one or more Musky ingredients and one or more Floral ingredients; or
one or more Musky ingredients, one or more Powdery ingredients, and one or more Floral ingredients;
   wherein the total amount of Musky ingredients, when present, is at least 1%, typically at least 2%, more typically at least 4%;
   the total amount of Powdery ingredients, when present, is at least 0.5%, typically at least 0.6%, more typically at least 0.8%; and
   the total amount of Floral ingredients, when present, is at least 10%, typically at least 15%, more typically at least 19%;
   wherein the total amount of Musky ingredients, Powdery ingredients, and Floral ingredients is at least 20%, typically at least 25%, more typically at least 30%, the amounts expressed by weight relative to the total weight of the composition; wherein the Musky, Powdery, and/or Floral ingredients are described herein.

The nature and concentrations of the ingredients already described with respect to the method and the mental performance enhancing composition or consumer product used in the method is applied mutatis mutandis to the aforementioned mental performance enhancing composition.

In the third aspect, the present disclosure relates to a consumer product comprising the mental performance enhancing composition described herein.

The form of the consumer product is not particularly limited. In some aspects, the consumer product is typically a perfumed consumer product. In an embodiment, the consumer product is a perfume, a fabric care product, a body-care product, a cosmetic preparation, a skin-care product, an air care product, or a home care product.

In another embodiment, the consumer product is a fine perfume, a splash, eau de parfum, a cologne, a shave or after-shave lotion, a liquid or solid or unit-dose detergent, a fabric softener, a solid or liquid fabric scent-booster, a fabric refresher, an ironing water, a paper, a bleach, a carpet cleaners, curtain-care products, a shampoo, a leave-on or rinse-off hair conditioner, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, or a foot/hand care products, a hygiene product, an air freshener, a “ready to use” powdered air freshener, a mold remover, a furniture care product, a wipe, a dish detergent or hard-surface detergent, a leather care product, or a car care product.

The amount of the mental performance enhancing composition in the consumer product is not particularly limited. A person of ordinary skill in the art would be able adjust the amount of the mental performance enhancing composition in the consumer product to deliver the effective amount of the mental performance enhancing composition to a subject or consumer so long as the composition is perceivable by the subject or consumer. The ordinarily skilled artisan would understand that the amount of the mental performance enhancing composition that would be perceivable to a subject or consumer would depend on the consumer product.

However, in some embodiments, the consumer product comprises from 0.1% to 85% by weight of the mental performance enhancing composition, relative to the total weight of the consumer product. In some embodiments, the consumer product comprises from 0.1% to 15%, typically 0.1% to 7%, more typically 0.1% to 5%, by weight of the mental performance enhancing composition, relative to the total weight of the consumer product. In some embodiments, the consumer product comprises from 0.1% to 2%, typically 0.1% to 0.5%, more typically 0.1% to 0.3%, by weight of the mental performance enhancing composition, relative to the total weight of the consumer product.

In some embodiments, the consumer product comprises from 0.5% to 15%, typically 0.5% to 7%, more typically 0.5% to 5%, by weight of the mental performance enhancing composition, relative to the total weight of the consumer product. In some embodiments, the consumer product comprises from 0.5% to 2% by weight of the mental performance enhancing composition, relative to the total weight of the consumer product.

In some embodiments, the consumer product comprises from 30% to 85%, typically 30% to 70%, more typically 30% to 60%, by weight of the mental performance enhancing composition, relative to the total weight of the consumer product. In some embodiments, the consumer product comprises from 40% to 85%, typically 40% to 70%, more typically 40% to 60%, by weight of the mental performance enhancing composition, relative to the total weight of the consumer product.

Consumer benefits, such as emotions, moods, feelings, mental states, or enhancement thereof, elicited by fragrances and aromas, typically over a period of time, are important drivers of consumer satisfaction. “Odor” or “smell” usually designates the perceived sensation elicited by one or several volatile chemicals—odorants—stimulating the olfactory organ. As a result of evolution, the olfactory system has a privileged access to emotion centers of the brain, providing odors with the ability to influence the human behavior, mood, and decisions.

In the fourth aspect, the present disclosure relates to a method for evaluating the ability of a material to deliver a consumer benefit over time, the method comprising conducting one or more sessions, wherein each session comprises:
a.) Exposing the subject to a material having an odor,
b.) Capturing the subject's perception of odor intensity,
c.) Optionally, allowing the subject to perform one or more tasks and measuring a parameter associated with each task,
d.) capturing the subject's mood or mental state,
e.) repeating steps a. to d. 1 to 10, typically 1 to 5, times.

In an embodiment, the consumer benefit is an emotion, mood, feeling, mental state, or enhancement thereof, typically enhancement of mental performance.

In an embodiment, the method comprises conducting two or more sessions, typically three or more sessions, wherein in at least one session, the subject is exposed to a material having no odor.

In an embodiment, each session is conducted 1 to 20 days, typically 1 to 15 days, more typically 1 to 10 days, apart.

In an embodiment, each repetition of steps a-d is 1 to 60 minutes, typically 1 to 30 minutes, more typically 1 to 20 minutes, from the previous repetition.

In an embodiment, each session is at least 30 minutes, typically at least 1 hour, more typically at least 2 hours.

In an embodiment, the task is a Stroop task or an arithmetic task.

In an embodiment, the parameter associated with each task is response time or accuracy.

Mention is made of the use of the mental performance enhancing composition or consumer product described herein to enhance the mental performance of a subject, typically a human being. In accordance with the present disclosure, the use of the mental performance enhancing composition or consumer product to enhance the mental performance of a subject is not therapeutic and is not meant to treat any disease or condition.

The compositions, products, uses, and methods according to the present disclosure are further illustrated by the following non-limiting examples.

### Example 1. Enhancement of Mental Performance

The ability of two mental performance enhancing compositions, one inventive composition (“Fragrance A”) and one comparative composition (“Fragrance B”), to enhance mental performance was evaluated. The mental performance enhancing compositions evaluated are summarized in Table 1 below.

TABLE 1

| | Fragrance A | Fragrance B |
|---|---|---|
| Powdery (wt %) | 2.06 | 0.002 |
| Musky (wt %) | 20.6 | 2.02 |
| Floral (wt %) | 4.1 | 3.6 |
| Total enhancing ingredients | 26.8 | 5.622 |
| Aromatic (wt %) | 0 | 0.2 |
| Citrus (wt %) | 0 | 78.4 |
| Spicy (wt %) | 2.1 | 0 |
| Total disrupting ingredients | 2.1 | 78.6 |

Mental performance during two different cognitive tasks was measured. One task is a Stroop task (see Stroop, J. R. (1935). Studies of interference in serial verbal reactions. Journal of Experimental Psychology, 18, 643-662, and MacLeod, C. M. (1991). Half a Century of Research on the Stroop Effect: An Integrative Review. Psychological Bulletin, 109 (2), 163-203), a classical paradigm to measure interference and attention, and the other task is a mental arithmetic task that measures quantitative reasoning in an ecologically valid setting.

Participants: 24 women (18-35 years old), non-smokers participated in the study after providing informed consent; they were paid for participation.

Tasks: Participants performed two mental performance tasks with the following instructions provided at the beginning of the experiment: “Today we will ask you to perform some mental performance tasks, please be aware that some fragrances might be diffused during the exercises.”
(1) In the Stroop Task, participants were asked to press one of three keys on the keyboard indicating the color (red, green, blue) in which a color word (red, green, blue) is displayed on the screen. The buttons on the keyboard were colored in red, green, blue, to make their identification easier. Color words and the color in which they are displayed could be matching (congruent) or mismatching (incongruent). The task lasted 3 minutes during which participants completed 155.3 trials on average (SD=13.5). 0.6% of trials were discarded from analysis because responses were not properly recorded.
(2) In the mental arithmetic task, participants performed a series of arithmetic operations with two different levels of difficulty: easy (summating 1- and 2-digit) and hard (summating 2- and 3-digit numbers). The easy task was always performed first. The task lasted 4 minutes, and participants completed 24.5 trials on average in the easy task (SD=3.3) and 14.5 on average in the hard task (SD=3.0). Response times (RTs) and accuracy were measured during the tasks. 0.04% of trials were discarded from analysis because responses were not properly recorded.

Odor conditions: The tasks were performed under three odor conditions: (a) with inventive “Fragrance A”, (b) with comparative “Fragrance B” (fragrance control), and (c) without odor (control condition). Both fragrances were diluted in dipropylene glycol (DIPG) at concentrations of 40% and 60%. The odors were diffused in separate cabins (140 cm×140 cm×197 cm) with a fragrance diffusor (UMA REF. 15207790, from Nature & Decouvertes) and participants moved between the three cabins. The order of odor presentation was balanced across participants.

Final questionnaire: At the end of the experiment, participants answered a set of questions to assess whether they perceived the fragrances and how intense and pleasant they were. Here, fragrances were presented via smelling strips to assess the extent of associated emotions while smelling using a known scale (see Porcherot, et al., Food Quality and Preference, Volume 21, Issue 8, December 2010, Pages 938-947). For this, nine emotional dimensions were rated on 101-point visual analogue scales (VAS) anchored with “no feeling” (0) and “very intense feelings” (100).

Statistical analyses: As indicators of task performance in the two cognitive tasks, RTs (response times) and accuracy of the responses were analyzed. The influence of task congruency (congruent, incongruent) or difficulty (easy, hard) on these indicators was assessed using Wilcoxon signed-rank tests, whereas the influence of the odor condition (“Fragrance A”, “Fragrance B”, control) was investigated using Friedman tests, followed by Wilcoxon signed-rank tests as post-hoc tests, if applicable. The comparison of the feelings (from final questionaire) elicited by the fragrances was analyzed with a one-way analysis of variance (ANOVA) with «fragrance» as within-subject factor. Posthoc tests were performed with Duncan's Multiple Range Test. The alpha level was set a priori to 0.05 for all statistical analyses.

### Results

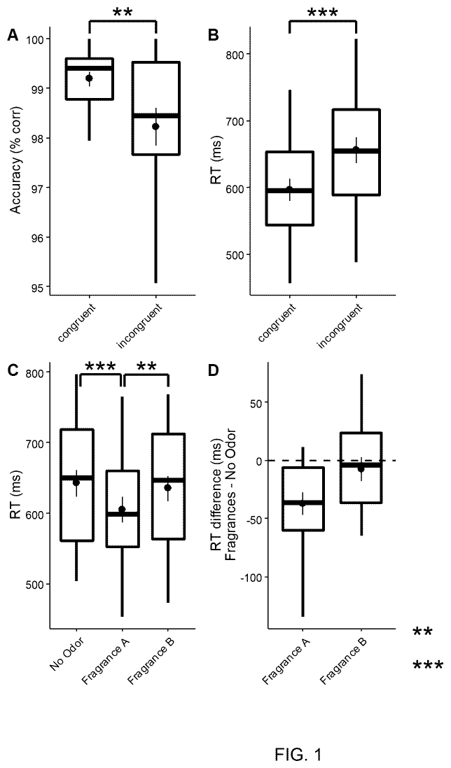

Stroop task: As shown in FIG. 1A, accuracy was higher in congruent compared to non-congruent trials [V=250, Z=2.95, p=0.003, r=0.60]. Accuracy was not influenced by the odor condition

$$[ {X}_{F}^{2} ( 2 ) = 0.89 , p = \text{.642} , w = 0.02 ] .$$

Replicating the Stroop effect, participants responded faster in congruent compared to incongruent trials [V=0, Z=5.29, p<0.001, r=1.08; FIG. 1B]. As shown in FIG. 1C and FIG. 1D, RTs varied significantly between odor conditions

$$[ {X}_{F}^{2} ( 2 ) = 14.25 , p < \text{.001} , w = 0.3 ]$$

faster responses, indicating increased mental processing speed, in the presence of “Fragrance A” compared to control [V=36, Z=3.45, p<0.001, r=0.70]and “Fragrance B” [V=45, Z=3.12, p=0.002, r=0.64]. Conversely, no significant difference was observed in presence of “Fragrance B” compared to control [V=136, Z=0.38, p=0.705, r=0.08]. Erroneous trials, implausible responses (i.e., RT<300 ms and RT>1500 ms), and responses falling outside 2.5 standard deviations from the participant's mean were discarded from analysis leading to the removal of 5.0% of trials.

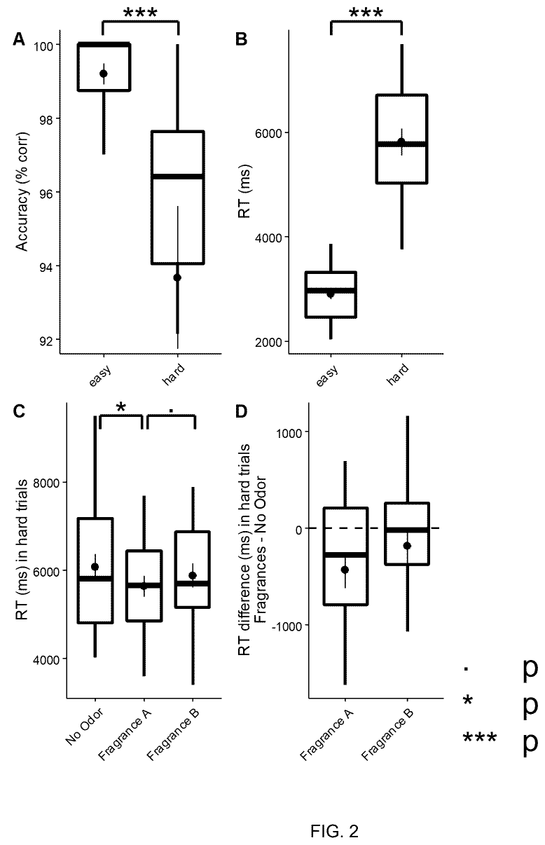

Arithmetic task: As expected, accuracy was higher in the easy compared to hard task [V=250, Z=3.99, p<0.001, r=0.82]as shown in FIG. 2A. Accuracy was not influenced by the odor condition

$$[ {X}_{F}^{2} ( 2 ) = 3.34 , p = \text{.188} , w = 0.07 ] .$$

Also, as expected, participants responded faster in the easy compared to hard task [V=0, Z=5.17, p<0.001, r=1.05]as shown in FIG. 2B. In the hard task, as shown in FIG. 2C and FIG. 2D, RTs marginally varied between odor conditions

$$[ {X}_{F}^{2} ( 2 ) = 5.48 , p = \text{.065} , w = 0.12 ] .$$

Specifically, “Fragrance A” showed significantly shorter RTs compared to control [V=70, Z=2.07, p=0.038, r=0.42]and marginally shorter RTs compared to “Fragrance B” [V=82, Z=1.69, p=0.092, r=0.34]. Conversely, no significant difference was observed in presence of “Fragrance B” compared to control [V=114, Z=0.70, p=0.482, r=0.14]. No odor effect was observed in the easy task

$$[ {X}_{F}^{2} ( 2 ) = 1.39 , p = \text{.499} , w = 0.03 ] .$$

Erroneous trials, implausible responses (i.e., RT<1500 ms and RT>12000 ms) and responses falling outside 2.5 standard deviations from the participant's mean were discarded from analysis leading to the removal of 5.8% of trials. One participant was excluded from the analysis because of an insufficient number of trials (i.e., more than 50% of their trials were discarded following RT preprocessing).

Final Questionnaire: Participants reported that they perceived a fragrance in the two odor conditions and found the experience pleasant. They perceived no fragrance in the control condition.

Together, the results show a significant decrease of RTs in both the Stroop and the hard arithmetic tasks indicating an increase of mental processing speed while keeping accuracy constant. Notably the effect was only observed in the presence of “Fragrance A” but not for “Fragrance B”. In sum, the data confirm that a fragrance specifically designed according to the present disclosure enhances mental performance and the effects can be captured with implicit and objective measures.

### Example 2. Exemplary Inventive Compositions

TABLE 2

| | Ex. C | Ex. D |
|---|---|---|
| Powdery (wt %) | 5 | 0.3 |
| Musky (wt %) | 8.3 | 85.3 |
| Floral (wt %) | 73.3 | 12 |
| Total enhancing ingredients | 86.6 | 97.6 |
| Aromatic (wt %) | 0 | 0.1 |
| Citrus (wt %) | 0 | 0 |
| Spicy (wt %) | 0 | 0 |
| Total disrupting ingredients | 0 | 0.1 |

TABLE 3

| | Ex. E | Ex. F |
|---|---|---|
| Powdery (wt %) | 16 | 0 |
| Musky (wt %) | 5.1 | 0 |
| Floral (wt %) | 8.2 | 30.3 |
| Total enhancing ingredients | 29.3 | 30.3 |
| Aromatic (wt %) | 0.1 | 0 |
| Citrus (wt %) | 0 | 0 |
| Spicy (wt %) | 0 | 0 |
| Total disrupting ingredients | 0.1 | 0 |

### Example 3. Enhancement of Mental Performance

Four inventive compositions (“Fragrance G”, “Fragrance H”, “Fragrance I” and “Fragrance J”) were evaluated for their potential to enhance mental performance. They are characterized in Tables 4 and 5.

TABLE 4

| | Fragrance G | Fragrance H |
|---|---|---|
| Powdery (wt %) | 0 | 25.0 |
| Musky (wt %) | 0 | 15.6 |
| Floral (wt %) | 91.7 | 52.5 |
| Total enhancing ingredients | 91.7 | 93.1 |
| Aromatic (wt %) | 0.3 | 0 |
| Citrus (wt %) | 0 | 0 |
| Spicy (wt %) | 0.7 | 0 |
| Total disrupting ingredients | 1.0 | 0 |

TABLE 5

| | Fragrance I | Fragrance J |
|---|---|---|
| Powdery (wt %) | 0.33 | 4.98 |
| Musky (wt %) | 84.06 | 5.78 |
| Floral (wt %) | 13.5 | 12.14 |
| Total enhancing ingredients | 97.9 | 22.9 |
| Aromatic (wt %) | 0.11 | 1.16 |
| Citrus (wt %) | 0 | 0 |
| Spicy (wt %) | 0.002 | 0.34 |
| Total disrupting ingredients | 0.11 | 1.5 |

The method and the procedure were identical to those used in Example 1, with the exception that five odor conditions were used.

Participants: 60 women (18-40 years old), non-smokers participated in the study after providing informed consent; they were paid for participation.

Tasks: participants performed the mental performance tasks with the following instruction provided at the beginning of the experiment: “Today we will ask you to perform some mental performance tasks, please be aware that some fragrances might be diffused during the exercises.” The Stroop task lasted 3 minutes during which participants completed 151.9 trials on average (SD=13.2). 0.6% of trials were discarded from analysis because responses were not properly recorded. The mental arithmetic task lasted 4 minutes during which participants competed 26.3 trials on average in the easy task (SD=2.3) and 15.5 on average in the hard task (SD=3.2). 0.1% of trials were discarded from analysis because responses were not properly recorded.

Odor conditions: The different tasks were performed under five odor conditions: (a) with inventive “Fragrance G”, (b) with inventive “Fragrance H”, (c) with inventive “Fragrance I”, (d) with inventive “Fragrance J” and (e) without odor (control condition). Fragrances were diluted in DIPG at concentrations of 30%, 40%, 70%, and 85%. Similar to Example 1, odors were diffused in separate cabins (140 cm×140 cm×197 cm) with a fragrance diffusor (UMA REF. 15207790, from Nature et Decouvertes) and participants moved between the five cabins. The order of odor presentation was balanced across participants.

Statistical analyses: Consistently with the approach used in Example 1, RTs (response times) and accuracy of the responses were analyzed separately. The influence of task congruency (congruent, incongruent) or difficulty (easy, hard) on these indicators was assessed using Wilcoxon signed-rank tests. Similarly, the impact of fragrances (“Fragrance G”, “Fragrance H”, “Fragrance I”, “Fragrance J”) was investigated using Wilcoxon signed-rank tests, comparing each Fragrance to the control condition.

### Results

We replicated the main effects observed in Example 1 for the majority of fragrances.

Stroop task: Accuracy was higher in congruent compared to non-congruent trials [V=1518, Z=4.77, p<0.001, r=0.62]. Accuracy was not impacted by the fragrances [all p. values>0.4].

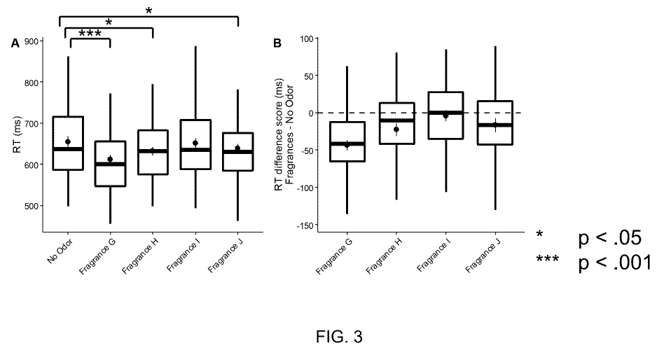

Participants responded faster in congruent compared to incongruent trials [V=0, Z=6.73, p<0.001, r=0.87]. As shown in FIG. 3A and FIG. 3B, RTs also critically varied between odor conditions with faster responses, indicating increased mental processing speed in the presence of “Fragrance G” [V=205, Z=5.22, p<0.001, r=0.67], “Fragrance H” [V=622, Z=2.15, p=0.031, r=0.28]and “Fragrance J” [V=612, Z=2.23, p<0.026, r=0.29]as compared to control. No significant difference was observed in presence of “Fragrance I” compared to control [V=868, Z=0.34, p=0.732, r=0.04]. Erroneous trials, implausible responses, and responses falling outside 2.5 standard deviations from the participant's mean were discarded from analysis leading to the removal of 6.5% of trials.

Arithmetic task: As expected, accuracy was higher in the easy compared to hard task [V=1561, Z=6.22, p<0.001, r=0.80]. Accuracy was not impacted by the fragrances [all p. values>0.7].

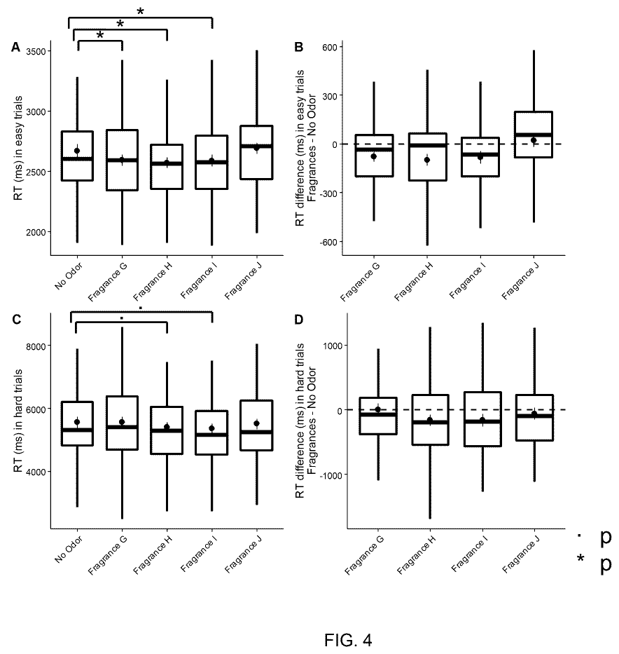

Participants responded faster in the easy compared to hard task [V=0, Z=6.73, p<0.001, r=0.87]. In the easy task, as shown in FIG. 4A and FIG. 4B, shorter RTs were observed in presence of “Fragrance G” [V=632, Z=2.08, p=0.038, r=0.27], “Fragrance H” [V=636, Z=2.05, p=0.040, r=0.26]and “Fragrance I” [V=600, Z=2.32, p=0.021, r=0.30]compared to control; but not in presence of “Fragrance J” [V=1099, Z=1.35, p=0.177, r=0.17]. In the hard task, as shown in FIG. 4C and FIG. 4D, shorter RTs were marginally observed in presence of “Fragrance H” [V=653, Z=1.93, p=0.054, r=0.25]and “Fragrance I” [V=602, Z=1.78, p=0.075, r=0.23]compared to control. Conversely, no difference was observed in presence of “Fragrance G” [V=811, Z=0.76, p=0.446, r=0.10]and “Fragrance J” [V=762, Z=1.12, p=0.262, r=0.14]compared to control. Erroneous trials, implausible responses, and responses falling outside 2.5 standard deviations from the participant's mean were discarded from analysis leading to the removal of 6.2% of trials. Three participants were excluded from hard task performed under “Fragrance I” condition because more than 50% of their trials were discarded during RT preprocessing.

Final Questionnaire: Participants reported that they perceived a fragrance in all four odor conditions and that they found the experience pleasant. As expected, they perceived no fragrance in the control condition.

To conclude, all four inventive fragrances showed a significant decrease of RTs in at least one condition of the Stroop or arithmetic task, indicating an increase of mental processing speed while keeping accuracy constant. Similar to Example 1, the data confirm that a fragrance specifically designed according to the present disclosure enhances mental performance and the effects can be captured with implicit and objective measures.

### Example 4. Sustained Enhancement of Mental Performance Over Time

Two inventive compositions (“Fragrance A”, “Fragrance K”) were evaluated for their potential to enhance mental performance during sustained mental activity over a period of two hours.

Participants: 60 women (18-40 years old, non-smokers) participated in the study after providing informed consent; they were paid for participation. Five participants were excluded from the analysis: one due to data acquisition issues and four due to non-compliance with the instructions of the Stroop task. The analysis was conducted on the remaining 55 participants.

Experimental design: The study consisted of three sessions with a one-week interval in between sessions. Each session lasted for two hours and involved 5 runs during which participants rated odor intensity, completed the Stroop task, and ratings scales under one of three odor conditions. Participants engaged in controlled activities such as reading and coloring mandalas during 20 minutes between each of the five runs. Before each Stroop task, participants evaluated the intensity of the odor. After each Stroop task, they completed a questionnaire with ten scales capturing their mental state and moods during the Stroop task. Only the scales on focus (i.e., alert, active, attentive) were deemed relevant and considered for the statistical analysis. The remaining seven scales were included to hide the purpose of the questionnaire. All scales included a 101-point VAS anchored with “not at all” (0) and “extremely” (100). Each Stroop task reiteration lasted 3 minutes during which participants completed 154.5 trials on average (SD=13.0). 0.6% of trials were discarded from analysis because responses were not properly recorded.

Odor conditions: The three sessions were performed under three odor conditions: (a) with inventive “Fragrance A”, (b) with inventive “Fragrance K” and (c) without odor (control condition). Both fragrances were diluted at 15% in a hydroalcholic solution tailored for use in fine fragrances. The fragrances were applied onto glass slides, which gives an indication of evaporative behavior of fragrances from surfaces, such as human skin. Glass slides were mounted onto a harmonica neck holder (K&M 164/15) which was worn by participants for the whole duration of the session. The order of odor presentations was balanced across participants.

Statistical analyses: Consistently with the approach used in Examples 1 and 3, RTs (response times) and accuracy of the responses from the Stroop task were analyzed separately. In contrast to Examples 1 and 3, parametric statistical tests were used to accommodate the multifactorial design, which could not be captured with non-parametric tests. RTs and accuracy were submitted to repeated-measured ANOVA with congruency (congruent, incongruent), repetitions (R1, R2, R3, R4, R5), and odor condition (“Fragrance A”, “Fragrance K”, control) as within-subject factors. The three ratings measuring subjective focus (alert, active, attentive) were combined into a single composite score justified by strong correlations (Spearman coefficients exceeding 0.70). The resulting composite score was then submitted to a repeated-measures ANOVA with repetitions (R1, R2, R3, R4, R5) and odor condition (“Fragrance A”, “Fragrance K”, control) as within-subject factors. Greenhouse-Geisser correction was applied to address potential violations of the sphericity assumption. Relevant post-hoc contrasts were performed using Student t-tests.

### Results

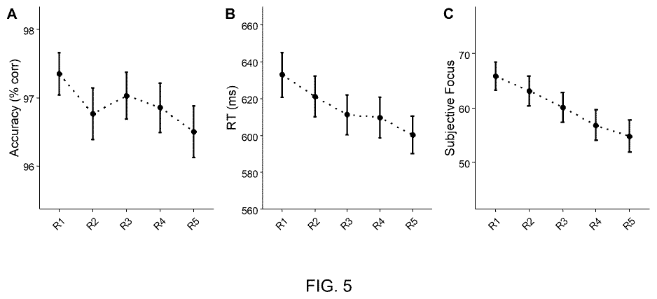

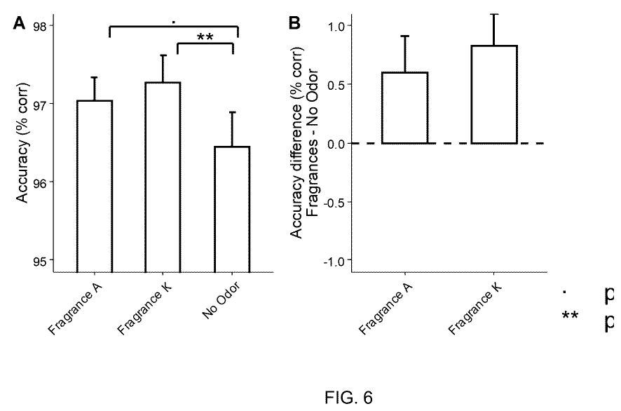

Stroop task: Replicating the findings observed in Examples 1 and 3, accuracy was higher in congruent compared to non-congruent trials [F(1, 54)=45.74, p<0.001, η²ₚ=0.46, 90% Cl [0.29, 0.57]]. As shown in FIG. 5A, accuracy significantly decreased across the five repetitions [F(3.48, 188.02)=5.83, p<0.001, η²ₚ=0.10, 90% Cl [0.03, 0.15]]; these findings is consistent with fatigue due to repeated measurements. Accuracy was significantly impacted by the odor condition [F(1.92, 103.80)=4.14, p=0.020, η²ₚ=0.07, 90% Cl [0.01, 0.15]; FIGS. 6A and 6B], with significantly higher accuracy while performing the task with “Fragrance K” [t(54)=3.10, p=0.003, d_{z}=0.42, 95% Cl [0.14, 0.69]]and marginally higher accuracy with “Fragrance A” [t(54)=1.87, p=0.067, d_{z}=0.25, 95% Cl [−0.02, 0.52]], compared to control. The interaction between repetitions and odor condition was not significant [F(6.25, 337.75)=1.37, p=0.224, η²ₚ=0.02, 90% Cl [0.00, 0.04]]indicating that the benefit provided by the inventive fragrances on mental performance was consistent over the testing period of two hours. No further interaction was significant.

As expected in the Stroop task, participants responded significantly faster in congruent compared to non-congruent trials [F(1, 54)=214.43, p<0.001, η²ₚ=0.80, 90% Cl [0.71, 0.84]]. As shown in FIG. 5B, RTs significantly decreased across the five repetitions [F(2.93, 158.16)=30.13, p<0.001, η²ₚ=0.36, 90% Cl [0.25, 0.43]]consistent with a learning effect. RTs were not significantly impacted by the odor conditions [F(1.94, 104.98)=2.07, p=0.133, η²ₚ=0.04, 90% Cl [0.00, 0.10]]nor were significant interactions observed. Erroneous trials, implausible responses, and responses falling outside 2.5 standard deviations from the participant's mean were discarded from analysis leading to the removal of 7.1% of trials.

Perceived intensity Participants reported that they clearly perceived a fragrance in the two odor conditions, across the five repetitions.

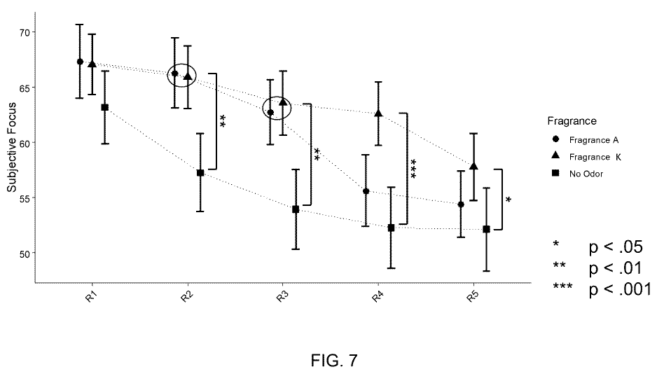

Subjective focus: As shown in FIG. 5C, subjective focus scores significantly decreased over the five repetitions [F(1.99, 107.35)=24.28, p<0.001, η²ₚ=0.31, 90% Cl [0.19, 0.41]]indicating fatigue. A significant interaction effect between repetitions and odor condition [F(5.67, 306.24)=2.66, p=0.018, η²ₚ=0.05, 90% Cl [0.00, 0.07]; FIG. 7]signifies significantly higher focus scores for “Fragrance K” in runs 2, 3, 4, and 5 (repetitions R2 [t(54)=3.05, p=0.004, d_{z}=0.41, 95% Cl [0.13, 0.68]], R3 [t(54)=3.78, p<0.001, d_{z}=0.51, 95% Cl [0.23, 0.79]], R4 [t(54)=3.96, p<0.001, d_{z}=0.53, 95% Cl [0.25, 0.81]], and R5 [t(54)=2.02, p=0.048, d_{z}=0.27, 95% Cl [0.00, 0.54]]as well as significantly higher focus scores for “Fragrance A” in runs 2 and 3 (repetitions R2 [t(54)=3.09, p=0.003, d_{z}=0.42, 95% Cl [0.14, 0.69]], and R3 [t(54)=3.16, p=0.003, d_{z}=0.43, 95% Cl [0.15, 0.70]]compared to control.

In summary, the application of inventive fragrances resulted in improved accuracy in the Stroop task that was repeated five times during a two-hour period. Importantly, RTs were not significantly affected evidencing an overall enhanced task performance. Inventive fragrances also were found to enhance the feeling of being focused over time. The data demonstrate that a fragrance designed according to the present disclosure enhance mental performance during sustained and repeated mental activity, and the effects are evident in both subjective and objective measures.

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

What is claimed is:

1. A method for enhancing mental performance of a subject, the method comprising:
a) providing a mental performance enhancing composition or a consumer product comprising the said mental performance enhancing composition,
b) exposing the subject, typically a human being, to the mental performance enhancing composition or the consumer product in an amount effective to enhance the mental performance of the subject;
wherein the mental performance enhancing composition comprises one or more Musky ingredients, one or more Powdery ingredients, one or more Floral ingredients, or any combination thereof,
wherein the total amount of Musky ingredients, when present, is at least 1%, typically at least 2%, more typically at least 4%;
the total amount of Powdery ingredients, when present, is at least 0.5%, typically at least 0.6%, more typically at least 0.8%; and
the total amount of Floral ingredients, when present, is at least 10%, typically at least 15%, more typically at least 19%;
wherein the total amount of Musky ingredients, Powdery ingredients, and Floral ingredients is at least 20%, typically at least 25%, more typically at least 30%, the amounts expressed by weight relative to the total weight of the composition;
wherein Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE,
(+−)-1-(3,5,5,6,8,8-hexamethyl-5,6,7,8-tetrahydro-2-naphthalenyl)ethenone,
(+−)-(4E)-3-METHYL-4-CYCLOPENTADECEN-1-ONE,
(+−)-3-METHYL-5-CYCLOPENTADECEN-1-ONE,
2-{(1 RS)-1-[3,3-dimethylcyclohexyl]ethoxy}-2-oxoethyl propionate,
2-oxo-2-{[2,6,6-trimethylcycloheptyl]oxy}ethyl propionate,
(+−)-4,6,6,7,8,8-hexamethyl-1,3,4,6,7,8-hexahydrocyclopenta[g]isochromene,
1-OXA-12-CYCLOHEXADECEN-2-ONE,
1-OXA-13-CYCLOHEXADECEN-2-ONE,
(9Z)-9-cycloheptadecen-1-one,
CYCLOPENTADECANONE,
oxacyclohexadecan-2-one,
1,4-dioxacycloheptadecane-5,17-dione,
(10E)-oxacycloheptadec-10-en-2-one,
(Z)-4-CYCLOPENTADECEN-1-ONE,
(6RS, 7RS)-3,5,5,6,7,8,8-heptamethyl-5,6,7,8-tetrahydro-2-naphthalenecarbaldehyde,
1,4-dioxacyclohexadecane-5,16-dione,
(1S)-1-[(1R)-3,3-dimethylcyclohexyl]ethyl [3-ethyl-2-oxiranyl]acetate,
(+−)-3-methylcyclopentadecanone,
AMBRETTE SEED ABSOLUTE, and any combination thereof,
Powdery ingredients are selected from the group consisting of:
TONKA ABSOLUTE,
VANILLA BOURBON,
VANILLE TAHITENSIS,
(3RS,3aRS,6SR,7aSR)-3,6-dimethylhexahydro-1-benzofuran-2(3H)-one,
(3RS,3aSR,6RS,7aRS)-3,6-dimethylhexahydro-1-benzofuran-2(3H)-one,
2-ETHOXY-4-METHYLPHENOL,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde,
2-CHROMENONE,
2-methoxy-4-methylphenyl methyl carbonate,
3,4-dimethoxybenzaldehyde,
4-(4-hydroxyphenyl)-2-butanone,
1,4-dimethoxybenzene,
(1 RS,2RS,7SR,8RS,9Z)-9-ethylidene-3-oxatricyclo[6.2.1.0~2,7~]undecan-4-one,
(1 RS,2RS,7SR,8SR,10Z)-10-ethylidene-3-oxatricyclo[6.2.1.0~2,7~]undecan-4-one,
(1 RS,2RS,7SR,8RS,9E)-9-ethylidene-3-oxatricyclo[6.2.1.0~2,7~]undecan-4-one,
(1 RS,2SR,7SR,8SR,10E)-10-ethylidene-3-oxatricyclo[6.2.1.0~2,7~]undecan-4-one,
FOIN ABS,
(+−)-4-hexanolide,
LIATRIX ABS TYPE,
4-(1,3-benzodioxol-5-yl)-2-butanone,
4-(4-hydroxyphenyl)-2-butanone,
VANILLE MADAGASCAR SFE 25 TEC,
VANILLE SURABSOLUE INCOLORE,
4-hydroxy-3-methoxybenzaldehyde,
and any combination thereof; and
Floral ingredients are selected from the group consisting of:
METHYL N-(7-HYDROXY-3,7-DIMETHYL-1-OCTENYL)ANTHRANILATE,
CANANGA OIL,
(2E,6E)-3,7,11-trimethyl-2,6,10-dodecatrien-1-ol,
FLEUR D ORANGER ABS,
ORANGE FLOWERS,
methyl 3,7-dimethyl-2,6-octadienoate,
GERANIUM ROSE OIL,
IRIS RESINOIDE,
JASMIN EGYPTE INFUSION,
JASMIN SAMBAC ABS CHINE PERF,
JASMINE OIL,
JASMIN INDES ABS SUPER,
METHYL N-[3-(4-TERT-BUTYLPHENYL)-2-METHYL-1-PROPENYL]ANTHRANILATE,
LILAC FIRBEST,
methyl 2-({[3-(4-hydroxy-4-methylpentyl)-3-cyclohexen-1-ylidene]methyl}amino)benzoate,
methyl 2-({[4-(4-hydroxy-4-methylpentyl)-3-cyclohexen-1-ylidene]methyl}amino)benzoate,
MICHELIA ALBA OIL,
MATE ABS,
MIMOSA PAYS ABS,
NARCISSUS ABS,
C6-12 ETHOXYLATED ALCOHOLS,
NEROLI EO,
OSMANTHUS ABS NAT,
PETITGRAIN ITALY EO,
PETITGRAIN S TERP,
ROSE ABS BULGARE,
TEA BLACK CEYLAN EXTR,
(+−)-2-propylheptanenitrile,
(2E)-2-METHYL-3-(4-METHYLPHENYL)-2-PROPEN-1-OL,
TUBEROSE INDIA ABS,
YLANG VOP,
1-(3-METHYL-1-BENZOFURAN-2-YL)ETHANONE,
1-(4-methylphenyl)ethenone,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
2-HEXYL-2-CYCLOPENTEN-1-ONE,
3-(4-TERT-BUTYLPHENYL)PROPANAL,
3,7-DIMETHYL-6-OCTEN-1-OL,
3,7-DIMETHYL-7-OCTEN-1-OL,
(+−)-2,4-dimethyl-4,4a,5,9b-tetrahydroindeno[1,2-d][1,3]dioxine,
ETHYL BENZOATE,
ETHYL PHENYLACETATE,
(+−)-(E)-3-METHYL-4-(2,6,6-TRIMETHYL-2-CYCLOHEXEN-1-YL)-3-BUTEN-2-ONE,
2-PHENYLETHANOL,
4-methyl-2-(2-methyl-1-propen-1-yl)tetrahydro-2H-pyran,
4-methyl-2-phenyltetrahydro-2H-pyran,
(3E)-4-(2,6,6-trimethyl-1-cyclohexen-1-yl)-3-buten-2-one,
(−)-(2S,4R)-4-methyl-2-(2-methyl-1-propen-1-yl)tetrahydro-2H-pyran,
1-(2-naphthyl)ethenone,
(4E)-4-methyl-5-(4-methylphenyl)-4-pentenal,
TRICYCLO[5.2.1.0(2,6)]DEC-3-EN-8-YL PROPANOATE,
3-methyl-2-[(2Z)-2-penten-1-yl]-2-cyclopenten-1-one,
(+−)-(3E)-4-(2,6,6-trimethyl-2-cyclohexen-1-yl)-3-buten-2-one,
(3E)-4-(2,6,6-trimethyl-1-cyclohexen-1-yl)-3-buten-2-one,
(1E)-1-(2,6,6-trimethyl-1-cyclohexen-1-yl)-1-penten-3-one,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
1-METHOXY-4-METHYLBENZENE,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-3-methyl-5-phenyl-1-pentanol,
(+−)-3,7-dimethyl-1,6-octadien-3-ol,
(+−)-(E)-3-METHYL-4-(2,6,6-TRIMETHYL-2-CYCLOHEXEN-1-YL)-3-BUTEN-2-ONE,
(E)-1-(2,6,6-TRIMETHYL-2-CYCLOHEXEN-1-YL)-1-PENTEN-3-ONE,
(+−)-(E)-1-(2,2-DIMETHYL-6-METHYLENE-1-CYCLOHEXYL)-1-PENTEN-3-ONE,
(E)-1-(2,6,6-TRIMETHYL-1-CYCLOHEXEN-1-YL)-1-PENTEN-3-ONE,
4-methyl-6-phenyl-2-hexanol,
3,7-DIMETHYL-2,6-OCTADIEN-1-OL,
(+−)-3-(1,3-BENZODIOXOL-5-YL)-2-METHYLPROPANAL,
(+−)-(E)-TRANS-alpha-IRONE,
(+−)-(E)-CIS-alpha-IRONE,
(+−)-(E)-beta-IRONE,
3,7-DIMETHYL-1,6-NONADIEN-3-OL,
methyl [3-oxo-2-pentylcyclopentyl]acetate,
2-PHENYLETHYL ACETATE,
2-PHENYLETHYL FORMATE,
(3Z)-3-hexen-1-yl salicylate,
1,1′-oxydibenzene,
tricyclo[5.2.1.0~2,6-]dec-3-en-8-yl acetate,
tricyclo[5.2.1.0~2,6-]dec-4-en-8-yl acetate,
(+−)-TRANS-3-METHYL-4-NONANOLIDE,
(+−)-3,7-DIMETHYL-3-OCTANOL,
(+−)-(Z)-7-DECEN-4-OLIDE,
(+−)-3,7-DIMETHYL-6-OCTEN-1-OL,
2-PHENYLETHYL PHENYLACETATE,
(+−)-5-ETHYL-2-NONANOL,
(+)-METHYL (1R)-CIS-3-OXO-2-PENTYL-1-CYCLOPENTANEACETATE,
1-octanol,
benzyl acetate,
CYCLOHEXYL 2-HYDROXYBENZOATE,
(+−)-8-DECEN-5-OLIDE,
CYCLOHEXYLIDENE(PHENYL)ACETONITRILE,
(+−)-3-(4-isopropylphenyl)-2-methylpropanal,
METHYL BENZOATE,
2-pentylcyclopentanol,
7-HYDROXY-3,7-DIMETHYLOCTANAL,
(+−)-7-hydroxy-3,7-dimethyloctanal,
(+−)-(3-methoxy-2-methylpropyl)benzene,
(+−)-3,7-dimethyl-1-octen-3-ol,
methyl cyclopentylideneacetate,
2-PHENYLETHYL 2-HYDROXYBENZOATE,
1-(5,6,7,8-tetrahydro-2-naphthalenyl)ethenone,
(+−)-2,2,2-TRICHLORO-1-PHENYLETHYL ACETATE,
methyl 2-((1 RS,2RS)-3-oxo-2-pentylcyclopentyl)acetate,
(2Z)-2-phenyl-2-hexenenitrile,
1,3-NONANEDIYL DIACETATE,
TETRAHYDRO-3-PENTYL-4(2H)-PYRANYL ACETATE,
(+−)-3,7-DIMETHYL-6-OCTENYL PROPANOATE,
(Z)-3,7-DIMETHYL-2,6-OCTADIEN-1-OL,
2-ETHOXYNAPHTHALENE,
(E)-2-PENTYL-3-PHENYL-2-PROPENAL,
9-DECEN-1-OL,
2-PHENYLETHYL ISOBUTYRATE,
benzyl salicylate,
4-CYCLOHEXYL-2-METHYL-2-BUTANOL,
1-NONANOL,
3-METHYLBUTYL 2-PHENYLETHYL ETHER,
(+−)-3,7-dimethyl-3-octanyl acetate,
ISOBUTYL PHENYLACETATE,
methyl {(1RS,2RS)-3-oxo-2-[(2Z)-2-penten-1-yl]cyclopentyl}acetate,
(+−)-TETRAHYDRO-2-ISOBUTYL-4-METHYL-4(2H)-PYRANOL,
ISOBUTYL 2-HYDROXYBENZOATE,
(−)-(2S)-1-oxo-1-(2-propanyloxy)-2-propanyl 2,2-dimethylpropanoate,
1-dodecanol,
(+−)-2-phenyl-1-propanol,
2-METHYLBUTYL SALICYLATE,
PENTYL SALICYLATE,
3-METHYLBUTYL PHENYLACETATE,
2-METHYLBUTYL PHENYLACETATE,
3-pentyltetrahydro-2H-pyran-4-yl acetate,
1,1-DIMETHYL-2-PHENYLETHYL ACETATE,
3,7-DIMETHYL-6-OCTENYL ACETATE,
BENZYL PHENYLACETATE,
[trans-4-(2-propanyl)cyclohexyl]methanol,
[cis-4-(2-propanyl)cyclohexyl]methanol,
(2E)-2-benzylideneoctanal,
2-BENZYL-4,4,6-TRIMETHYL-1,3-DIOXANE,
3-[(1R)-4-methyl-3-cyclohexen-1-yl]-1-butanol,
CIS-2-BENZYL-1,3-DIOXAN-5-OL,
2-BENZYL-1,3-DIOXOLANE-4-METHANOL,
TRANS-2-BENZYL-1,3-DIOXAN-5-OL,
6,10-dimethyl-5,9-undecadien-2-one,
HEXYL 2-HYDROXYBENZOATE,
3,7,11-TRIMETHYL-1,6,10-DODECATRIEN-3-OL,
2-phenoxyethanol,
1-PHENYL-1-ETHANOL,
(E)-3,7-DIMETHYL-2,6-OCTADIENYL PROPANOATE,
2-METHYL-4-PHENYL-2-BUTANOL,
(+−)-2,4,6-TRIMETHYL-3-CYCLOHEXENE-1-METHANOL,
2-METHYL-1-PHENYL-2-PROPANOL,
2-phenoxyethyl 2-methylpropanoate,
decyl acetate,
(E)-3,7-dimethyl-2,6-octadienyl acetate,
(2Z)-3,7-dimethyl-2,6-octadien-1-yl acetate,
(+−)-3,7-dimethyloctyl acetate,
tricyclo[5.2.1.0~2,6-]dec-3-en-8-yl acetate,
tricyclo[5.2.1.0~2,6-]dec-4-en-8-yl acetate,
citronellyl acetate,
neryl acetate,
geranyl acetate,
3,5,5-trimethylhexyl acetate,
4-phenyl-2-butanone,
4-(2,6,6-trimethyl-1-cyclohexen-1-yl)-2-butanone,
(3E)-4-(2,6,6-trimethyl-1-cyclohexen-1-yl)-3-buten-2-one,
4-(1,3-benzodioxol-5-yl)-2-butanone,
CAPUCINE M SA,
CASSIE ABSOLUTE,
1-phenyl-2-(1-propoxyethoxy)ethane,
(+−)-8,8-dimethyl-1,2,3,4,5,6,7,8-octahydro-2-naphthalenecarbaldehyde,
(+−)-5,5-dimethyl-1,2,3,4,5,6,7,8-octahydro-2-naphthalenecarbaldehyde,
(+−)-2,2-dimethyltricyclo[6.2.1.0~1,6-]undecan-7-one,
(+−)-2-pentylcyclopentanone,
2,6-dimethyl-2-heptanol,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
(+−)-(4Z)-4,8-dimethyl-4,9-decadienal,
(+−)-(4E)-4,8-dimethyl-4,9-decadienal,
(+−)-3-(3-isopropyl-1-phenyl)butanal,
(+−)-3,7-dimethyl-6-octenyl formate,
(+−)-3,7-dimethyl-6-octen-1-yl formate,
(2E)-3,7-dimethyl-2,6-octadien-1-yl formate,
GARDENIA FIRABSOLUE,
thiodamascone,
(1Z)-2-methyl-1-undecen-1-yl 2-phenylethyl ether,
(1E)-2-methyl-1-undecen-1-yl 2-phenylethyl ether,
thioionone,
(+−)-4-(dodecylthio)-4-(2,6,6-trimethyl-2-cyclohexen-1-yl)-2-butanone,
(+−)-4-(dodecylthio)-4-(2,6,6-trimethyl-1-cyclohexen-1-yl)-2-butanone,
benzo[d][1,3]dioxole-5-carbaldehyde,
(+−)-(3E)-4-(2,6,6-trimethyl-2-cyclohexen-1-yl)-3-buten-2-one,
2-nonenenitrile,
cis-4-(2-methyl-2-propanyl)cyclohexyl acetate,
trans-4-(2-methyl-2-propanyl)cyclohexyl acetate,
MUGUET FIRBEST,
(+−)-(4Z)-5-cyclohexyl-2,4-dimethyl-4-pentenal,
(+−)-(4E)-5-cyclohexyl-2,4-dimethyl-4-pentenal,
ORENOL P SAD,
PALMAROSA,
PIVOINE BLANCHE FIRBEST,
benzyl propanoate,
(+−)-methyl 2,2-dimethyl-6-methylidenecyclohexanecarboxylate,
(−)-(3R)-3,7-dimethyl-1,6-octadien-3-ol,
(+−)-3,7-dimethyl-1-octanol,
TEA GREEN FIRBEST,
3-(4,4-dimethyl-1-cyclohexen-1-yl)propanal,
LINDEN FIRBEST,
VIOTRIL BASE,
PETITGRAIN PARAGUAY HE,
YLANG EXTRA INFUSION,
YLANG EXTRA FIRNAT,
3-[4-(2-hydroxy-2-methylpropyl)phenyl]propanal,
(+−)-2-[4-(2-hydroxy-2-methylpropyl)phenyl]propanal,
3-(3-(2-hydroxy-2-methylpropyl)phenyl)propanal,
and any combination thereof.

2. The method according to claim 1,
wherein the total amount of Musky ingredients, when present, is 1% to 95% typically from 2% to 80%, more typically from 4% to 70%;
the total amount of Powdery ingredients, when present, is from 0.5% to 95%, typically from 0.6% to 80%, more typically from 0.8% to 70%; and
the total amount of Floral ingredients, when present, is from 10% to 95%, typically from 15% to 80, more typically from 19% to 70%;
wherein the total amount of Musky ingredients, Powdery ingredients, and Floral ingredients is from 20% to 95%, typically from 25% to 80%, more typically from 30% to 70%.

3. The method according to claim 1 or 2, wherein the mental performance enhancing composition comprises
one or more Musky ingredients and one or more Powdery ingredients;
one or more Powdery ingredients and one or more Floral ingredients;
one or more Musky ingredients and one or more Floral ingredients; or
one or more Musky ingredients, one or more Powdery ingredients, and one or more Floral ingredients.

4. The method according to any one of claims 1-3, wherein the one or more Musky ingredients are selected from the group consisting of:
(6RS,7RS)-3,5,5,6,7,8,8-heptamethyl-5,6,7,8-tetrahydro-2-naphthalenecarbaldehyde and (1S)-1-[(1R)-3,3-dimethylcyclohexyl]ethyl [3-ethyl-2-oxiranyl]acetate;
the one or more Powdery ingredients are selected from the group consisting of:
VANILLE TAHITENSIS,
(3S,3aS,6R,7aR)-3,6-dimethylhexahydro-1-benzofuran-2(3H)-one,
1-oxaspiro[4.5]decan-2-one,
2-methoxy-4-methylphenyl methyl carbonate,
VANILLE MADAGASCAR SFE 25 TEC,
VANILLE SURABSOLUE INCOLORE, and
4-hydroxy-3-methoxybenzaldehyde;
the one or more Floral ingredients are selected from the group consisting of:
JASMIN EGYPTE INFUSION,
LILAC FIRBEST,
PETITGRAIN S TERP,
(+−)-2-propylheptanenitrile,
(2E)-2-METHYL-3-(4-METHYLPHENYL)-2-PROPEN-1-OL,
(4E)-4-methyl-5-(4-methylphenyl)-4-pentenal,
4-methyl-6-phenyl-2-hexanol,
(+−)-(Z)-7-DECEN-4-OLIDE,
(+)-METHYL (1R)-CIS-3-OXO-2-PENTYL-1-CYCLOPENTANEACETATE,
(−)-(2S)-1-oxo-1-(2-propanyloxy)-2-propanyl 2,2-dimethylpropanoate,
YLANG EXTRA INFUSION,
YLANG EXTRA FIRNAT,
or any combination thereof.

5. The method according to any one of claims 1-4, wherein the mental performance enhancing composition comprises:
from 0% to 5.5%, typically from 0% to 5%, more typically from 0% to 4.5%, of Aromatic ingredients,
from 0% to 3.5%, typically from 0% to 3%, more typically from 0% to 1% of Spicy ingredients,
from 0% to 5.5%, typically from 0% to 5%, more typically from 0% to 4.5%, of Citrus ingredients, or any combination thereof,
wherein the total amount of Aromatic ingredients, Spicy ingredients, and Citrus ingredients is from 0% to 10%, typically from 0% to 6%, the amounts expressed by weight relative to the total weight of the composition.

6. The method according to any one of claims 1-5, wherein the mental performance enhancing composition comprises:
from 0% to 1%, typically from 0% to 0.7%, more typically from 0% to 0.4%, of Aromatic ingredients,
from 0% to 0.1%, typically from 0% to 0.05%, more typically from 0% to 0.02% of Spicy ingredients,
from 0% to 5%, typically from 0% to 4%, more typically from 0% to 3%, of Citrus ingredients, or any combination thereof;
wherein the total amount of Aromatic ingredients, Spicy ingredients, and Citrus ingredients is from 0% to 10%, typically from 0% to 5%, more typically from 0% to 3%, the amounts expressed by weight relative to the total weight of the composition;
wherein the Aromatic ingredients are selected from the group consisting of:
(+−)-1,7,7-trimethylbicyclo[2.2.1]heptan-2-one,
(−)-(5R)-5-isopropenyl-2-methyl-2-cyclohexen-1-one,
1,3,3-trimethyl-2-oxabicyclo[2.2.2]octane,
LAVANDIN GROSSO ARR,
5-methyl-2-(2-propanyl)cyclohexanone,
METHYL 2-HYDROXYBENZOATE,
thymol,
WORMWOOD OIL,
(+)-(1R,2R,4S)-1,3,3-trimethylbicyclo[2.2.1]heptan-2-ol,
4-methoxybenzaldehyde,
1-methoxy-4-[(1E)-1-propen-1-yl]benzene,
ANGELICA ROOT OIL,
METHYL 4-METHOXYBENZOATE,
ARTEMISIA OIL ARR,
BASIL OIL,
3-BUTYLIDENE-1-BENZO[C]FURANONE,
CAMOMILE,
ROMAN CHAMOMILE FLOWER OIL,
(+−)-2,2-DIMETHYL-3-METHYLENE-BICYCLO[2.2.1]HEPTANE,
alpha-PINENE,
CAMPHENE
EUCALYPTOL,
beta-PINENE,
SABINENE,
CARROTSEED OIL,
CARAWAY OIL,
(+)-(5S)-5-isopropenyl-2-methyl-2-cyclohexen-1-one,
CELERY SEED OIL,
(+−)-3-METHYL-5-PROPYL-2-CYCLOHEXEN-1-ONE,
1,4-EPOXY-P-MENTHANE,
1,8-EPOXY-P-MENTHANE,
CORIANDER OIL,
2,6-DIMETHYL-4-HEPTANOL,
1-METHOXY-4-PROPYLBENZENE,
(+−)-2,6-DIMETHYL-7-OCTEN-4-ONE,
1-methoxy-4-(2-propen-1-yl)benzene,
ESTRAGON OIL,
EUCALYPTUS BIOABSOLUTE,
FENNEL OIL BITTER,
FENUGREEK HYPERESSENCE,
(+−)-2-sec-butylcyclohexanone,
HYSSOP OIL,
EVERLASTING EO,
(−)-(1R,3R,4S)-8-P-MENTHEN-3-OL,
LAUREL LEAF OIL,
ASPIC NAT,
LAVENDER OIL,
LITSEA CUBEBA BERRY OIL,
MARJORAM EO,
PEPPERMINT OIL BRAZIL,
Peppermint USA Cascade OIL,
MENTHE POULIOT HE,
MINT SPICATA FW NATIVE,
(−)-L-MENTHOL,
(1 RS,3RS,4SR)-3-P-MENTHANOL,
MYRTLE OIL,
ORIGAN HE,
5-methyltricyclo[6.2.1.0~2,7~~]undecan-4-one,
REGLISSE ABS,
ROSEMARY EXTRACT,
2,2,8,10-tetramethylspiro[5.5]undec-8-en-1-one,
2,2,7,9-tetramethylspiro[5.5]undec-8-en-1-one,
DALMATIAN SAGE,
CLARY SAGE OIL,
4,5,6,7,8,9,10,11,12,13-DECAHYDROCYCLODODECA-1,3-OXAZOLE,
TAGETE EO,
2-methoxy-4-propylcyclohexanol,
(+−)-1-(3,3-dimethylcyclohexyl)ethenone,
(+−)-2,6,6-trimethylcycloheptanone,
(+−)-2-ethyl-4,4-dimethylcyclohexanone,
(1R,4S)-1,3,3-trimethylbicyclo[2.2.1]heptan-2-one,
1-isopropyl-4-methylbicyclo[3.1.0]hexan-3-one,
THYME RED EO,
1-(4-methoxyphenyl)-1-ethanone,
(+−)-1-pentyl-2-propenyl acetate,
4-methoxy-1-benzenemethanol,
STAR ANISE OIL,
BADIANE SFE EXTRACT,
5-isopropyl-2-methylphenol,
CEDARLEAF OIL,
5-methyl-3-heptanone,
(+−)-3-(4-methoxyphenyl)-2-methylpropanal,
WINTERGREEN OIL NAT,
2-butyl-4,4,6-trimethyl-1,3-dioxane,
methyl 2-hydroxybenzoate,
ethyl 2-hydroxybenzoate,
(−)-propyl (2S)-2-[(2-methyl-2-butanyl)oxy]propanoate,
SPEARMINT OIL ORG,
and any combination thereof,
the Spicy ingredients are selected from the group consisting of:
(E)-3-PHENYL-2-PROPENAL,
(−)-(1R,4E,9S)-4,11,11-trimethyl-8-methylenebicyclo[7.2.0]undec-4-ene,
2-methoxy-4-(2-propen-1-yl)phenol,
2-methoxy-4-[(1E)-1-propen-1-yl]phenol,
1,2-dimethoxy-4-[1-propen-1-yl]benzene,
4-(2-propanyl)benzaldehyde,
4-allyl-2-methoxyphenyl acetate,
(2E)-2-methyl-3-phenyl-2-propenal,
CASSIA OIL,
CINNAMON OIL CEYLON,
CARDAMOM OIL,
(E)-3-PHENYL-2-PROPENENITRILE,
2-methoxy-4-propylphenol,
ELEMI OIL,
4-ETHYL-2-METHOXYPHENOL,
GINGER,
ENGLISH CLOVE OIL,
GIROFLE TERPENES,
NUTMEG ABSOLUTE PURE AND NAT,
PIMENTA BERRY OIL,
BLACK PEPPER STEAM DIST.,
SICHUAN PEPPER (XANTHOXYLUM/ZANTHOXYLUM PIPERITUM),
(+−)-2,3,3-TRIMETHYL-1-INDANONE,
SAFRAN FIRBEST,
2,6,6-TRIMETHYL-1,3-CYCLOHEXADIENE-1-CARBALDEHYDE,
2-methoxy-4-vinylphenol,
4-isopropyl-1-benzenemethanol,
(1 RS,2SR,5RS,8SR)-4,4,8-trimethyltricyclo[6.3.1.0~2,5-]dodec-1-yl formate,
(1RS,2RS,5RS,8SR)-4,4,8-trimethyltricyclo[6.3.1.0~1,5-]dodec-2-yl formate,
CUMIN,
2-ethoxy-4-(methoxymethyl)phenol,
2,6,6-trimethyl-2-cyclohexene-1,4-dione,
turmeric oil,
and any combination thereof; and
Citrus ingredients are selected from the group consisting of:
(+−)-1,5-DIMETHYL-1-VINYL-4-HEXENYL ACETATE,
(+−)-2-(4-methyl-3-cyclohexen-1-yl)-2-propanyl acetate,
(−)-(R)-3,7-DIMETHYL-6-OCTENENITRILE,
trimethyl-4-phenyl-1,3-dioxane,
3,7-dimethyl-2,6-nonadienenitrile,
3,7-dimethyl-3,6-nonadienenitrile,
(+)-LIMONENE,
(4Z)-4-dodecenal,
(+)-(R)-3,7-DIMETHYL-6-OCTENAL,
3,7-DIMETHYL-2,6-OCTADIENAL,
ORANGE TERP CITRONOVA,
cis-4-isopropyl-1-methylcyclohexyl acetate,
2-(4-methylcyclohexyl)-2-propanyl acetate,
trans-4-isopropyl-1-methylcyclohexyl acetate,
2-(6,6-DIMETHYL-BICYCLO[3.1.1]HEPT-2-EN-2-YL)ETHYL ACETATE,
(+−)-3,7-dimethyl-1,6-nonadien-3-yl acetate,
1-METHYL-1 3-CYCLOHEXADIENE,
2-METHYL-1 3-CYCLOHEXADIENE,
3-METHYLENE-1-CYCLOHEXENE,
BERGAMOT FUROCOUMARIN-FREE,
BERGAMOTE ESS,
CEDRAT ESSENTIAL OIL,
LEMON OIL CALIF NAEFCO BRAND,
3-methyl-5-phenyl-2-pentenenitrile,
(+−)-2,6-DIMETHYL-7-OCTEN-2-OL,
4-(2-methoxy-2-propanyl)-1-methylcyclohexene,
GRAPEFRUIT ARR DECOL,
KAFFIR LIME LEAF OIL,
LIME OIL DIST,
LIME EXPR SSQT FAB,
LIME OIL TERPENES,
1,4/8-EPOXY-P-MENTHANE,
LIME OXIDE,
MANDARINE SFUMA,
6,6-DIMETHOXY-2,5,5-TRIMETHYL-2-HEXENE,
3,6,7-TRIMETHYL-2,6-OCTADIENAL,
(+)-(4R,4aS,6R)-4,4a-dimethyl-6-(1-propen-2-yl)-4,4a,5,6,7,8-hexahydro-2(3H)-naphthalenone,
ORANGE ESSENCE,
ORANGE COLORLESS ESSENTIAL OIL MOLECULAR DISTILLATION,
ORANGE TERP CITRONOVA,
PORTUGAL FLORIDE DECOL,
DECANAL,
DODECANAL,
beta-SINENSAL,
GERANIAL,
NERAL,
alpha-SINENSAL,
NOOTKATONE,
ELEMOL,
1,5,9-trimethyl-4,8-decadienyl acetate,
octyl acetate,
1-decanol,
(+−)-2-methyl-3-(4-methylphenyl)propanal,
(+)-(1 S,6R)-3,7,7-trimethylbicyclo[4.1.0]hept-3-ene,
(2Z)-1,1-dimethoxy-3,7-dimethyl-2,6-octadiene,
(2E)-1,1-dimethoxy-3,7-dimethyl-2,6-octadiene,
(2Z)-1,1-diethoxy-3,7-dimethyl-2,6-octadiene,
(2E)-1,1-diethoxy-3,7-dimethyl-2,6-octadiene,
LEMON CP FCR DECOL,
LEMON OIL DLE,
LEMON ESS 1341 ARR,
LEMON OIL WINTER TORCHIO DECOL,
CITRON SFUMA DECOL,
CITRON SFUMA,
Lemon oil Primofiore,
TETRAROME CITRON,
CITRONNELLE ESS BIO
CITRONELLA OIL,
CITRONELLA CEYLON EO,
methyl 2-(methylamino)benzoate,
2,6-dimethyl-7-octen-2-ol,
1,1,5-trimethyl-6-heptenyl formate,
alpha-terpinene,
(+−)-limonene,
alpha-terpinolene,
EUCALYPTUS CITRIODORA OIL,
EUCALYPTUS CITRIODORA ORGANIC EO,
LEMONGRASS,
(+−)-2-ethenyl-2,6,6-trimethyltetrahydro-2H-pyran,
(+)-(3S)-3-[(1R)-4-methyl-3-cyclohexen-1-yl]butanal,
(+)-(3R)-3-[(1R)-4-methyl-3-cyclohexen-1-yl]butanal,
LITSEA CUBEBA BERRY OIL,
(3Z)-3,12-tridecadienenitrile,
(3E)-3,12-tridecadienenitrile,
methyl 2-(methylamino)benzoate,
methyl octanoate,
ORANGE SANGUINE DM,
(+−)-3-methyl-5-phenylpentanal,
MELALEUCA,
(+−)-2,6-dimethyl-2-octanol,
(4E)-4-decenal,
(+−)-3,7-dimethyloctanal,
and any combination thereof.

7. The method according to any one of claims 1-6, wherein the total number of Musky ingredients, Powdery ingredients, and Floral ingredients is at least 3, typically at least 5, more typically at least 7.

8. The method according to any one of claims 1-7, wherein the one or more Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE], and any combination thereof;
the one or more Powdery ingredients are selected from the group consisting of:
3,6-dimethylhexahydro-1-benzofuran-2(3H)-one,
2-ETHOXY-4-METHYLPHENOL,
3-METHYL-4-OCTANOLIDE,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde, and any combination thereof;
the one or more Floral ingredients are selected from the group consisting of:
1-(3-METHYL-1-BENZOFURAN-2-YL)ETHANONE,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
3-(4-TERT-BUTYLPHENYL)PROPANAL,
3,7-DIMETHYL-6-OCTEN-1-OL,
3,7-DIMETHYL-7-OCTEN-1-OL,
(+−)-2,4-dimethyl-4,4a,5,9b-tetrahydroindeno[1,2-d][1,3]dioxine, and any combination thereof.

9. The method according to claim 8, wherein the one or more Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE], and any combination thereof;
the one or more Powdery ingredients are selected from the group consisting of:
3,6-dimethylhexahydro-1-benzofuran-2(3H)-one,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde, and any combination thereof;
the one or more Floral ingredients are selected from the group consisting of:
1-(3-METHYL-1-BENZOFURAN-2-YL)ETHANONE,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
3-(4-TERT-BUTYLPHENYL)PROPANAL,
3,7-DIMETHYL-6-OCTEN-1-OL,
3,7-DIMETHYL-7-OCTEN-1-OL, (+−)-2,4-dimethyl-4,4a,5,9b-tetrahydroindeno[1,2-d][1,3]dioxine, and any combination
thereof.

10. The method according to any one of claims 1-9, wherein the mental performance enhancing composition further comprises a phytoactive enhancer.

11. The method according to any one of claims 1-10, wherein the consumer product is a perfume, a fabric care product, a body-care product, a cosmetic preparation, a skin-care product, an air care product, or a home care product.

12. The method according to any one of claims 1-11, wherein the subject's reaction time in performing a task is reduced during or after exposure to the mental performance enhancing composition or a consumer product comprising the said mental performance enhancing composition.

13. A mental performance enhancing composition comprising:
one or more Musky ingredients and one or more Powdery ingredients;
one or more Powdery ingredients and one or more Floral ingredients;
one or more Musky ingredients and one or more Floral ingredients; or
one or more Musky ingredients, one or more Powdery ingredients, and one or more Floral ingredients;
wherein the total amount of Musky ingredients, when present, is at least 1%, typically at least 2%, more typically at least 4%;
the total amount of Powdery ingredients, when present, is at least 0.5%, typically at least 0.6%, more typically at least 0.8%; and
the total amount of Floral ingredients, when present, is at least 10%, typically at least 15%, more typically at least 19%;
wherein the total amount of Musky ingredients, Powdery ingredients, and Floral ingredients is at least 20%, typically at least 25%, more typically at least 30%, the amounts expressed by weight relative to the total weight of the composition;
wherein Musky ingredients are selected from the group consisting of:
(+)-2-{(1 S)-1-[(1R)-3,3-dimethylcyclohexyl]ethoxy}-2-methylpropyl propionate,
1-(4-TERT-BUTYL-3,5-DINITRO-2,6-DIMETHYLPHENYL)-1-ETHANONE,
(+−)-1-(3,5,5,6,8,8-hexamethyl-5,6,7,8-tetrahydro-2-naphthalenyl)ethenone,
(+−)-(4E)-3-METHYL-4-CYCLOPENTADECEN-1-ONE,
(+−)-3-METHYL-5-CYCLOPENTADECEN-1-ONE,
2-{(1 RS)-1-[3,3-dimethylcyclohexyl]ethoxy}-2-oxoethyl propionate,
2-oxo-2-{[2,6,6-trimethylcycloheptyl]oxy}ethyl propionate,
(+−)-4,6,6,7,8,8-hexamethyl-1,3,4,6,7,8-hexahydrocyclopenta[g]isochromene,
1-OXA-12-CYCLOHEXADECEN-2-ONE,
1-OXA-13-CYCLOHEXADECEN-2-ONE,
(9Z)-9-cycloheptadecen-1-one,
CYCLOPENTADECANONE,
oxacyclohexadecan-2-one,
1,4-dioxacycloheptadecane-5,17-dione,
(10E)-oxacycloheptadec-10-en-2-one,
(Z)-4-CYCLOPENTADECEN-1-ONE,
(6RS, 7RS)-3,5,5,6,7,8,8-heptamethyl-5,6,7,8-tetrahydro-2-naphthalenecarbaldehyde,
1,4-dioxacyclohexadecane-5,16-dione,
(1S)-1-[(1R)-3,3-dimethylcyclohexyl]ethyl [3-ethyl-2-oxiranyl]acetate,
(+−)-3-methylcyclopentadecanone,
AMBRETTE SEED ABSOLUTE, and any combination thereof,
Powdery ingredients are selected from the group consisting of:
TONKA ABSOLUTE,
VANILLA BOURBON,
VANILLE TAHITENSIS,
(3RS,3aRS,6SR,7aSR)-3,6-dimethylhexahydro-1-benzofuran-2(3H)-one,
(3RS,3aSR,6RS,7aRS)-3,6-dimethylhexahydro-1-benzofuran-2(3H)-one,
2-ETHOXY-4-METHYLPHENOL,
(3S,3aS,6R,7aR)-3,6-dimethylhexahydro-1-benzofuran-2(3H)-one,
PERHYDRO-2-CHROMENONE,
4-hydroxy-3-methoxybenzaldehyde,
1-oxaspiro[4.5]decan-2-one,
3-ethoxy-4-hydroxybenzaldehyde,
2-CHROMENONE,
2-methoxy-4-methylphenyl methyl carbonate,
3,4-dimethoxybenzaldehyde,
4-(4-hydroxyphenyl)-2-butanone,
1,4-dimethoxybenzene,
(1 RS,2RS,7SR,8RS,9Z)-9-ethylidene-3-oxatricyclo[6.2.1.0~2,7~]undecan-4-one,
(1 RS,2RS,7SR,8SR,10Z)-10-ethylidene-3-oxatricyclo[6.2.1.0~2,7~]undecan-4-one,
(1 RS,2RS,7SR,8RS,9E)-9-ethylidene-3-oxatricyclo[6.2.1.0~2,7~]undecan-4-one,
(1 RS,2SR,7SR,8SR,10E)-10-ethylidene-3-oxatricyclo[6.2.1.0~2,7~]undecan-4-one,
FOIN ABS,
(+−)-4-hexanolide,
LIATRIX ABS TYPE,
4-(1,3-benzodioxol-5-yl)-2-butanone,
4-(4-hydroxyphenyl)-2-butanone,
VANILLE MADAGASCAR SFE 25 TEC,
VANILLE SURABSOLUE INCOLORE,
4-hydroxy-3-methoxybenzaldehyde,
and any combination thereof; and
Floral ingredients are selected from the group consisting of:
METHYL N-(7-HYDROXY-3,7-DIMETHYL-1-OCTENYL)ANTHRANILATE,
CANANGA OIL,
(2E,6E)-3,7,11-trimethyl-2,6,10-dodecatrien-1-ol,
FLEUR D ORANGER ABS,
ORANGE FLOWERS,
methyl 3,7-dimethyl-2,6-octadienoate,
GERANIUM ROSE OIL,
IRIS RESINOIDE,
JASMIN EGYPTE INFUSION,
JASMIN SAMBAC ABS CHINE PERF,
JASMINE OIL,
METHYL N-[3-(4-TERT-BUTYLPHENYL)-2-METHYL-1-PROPENYL]ANTHRANILATE,
LILAC FIRBEST,
methyl 2-({[3-(4-hydroxy-4-methylpentyl)-3-cyclohexen-1-ylidene]methyl}amino)benzoate,
methyl 2-({[4-(4-hydroxy-4-methylpentyl)-3-cyclohexen-1-ylidene]methyl}amino)benzoate,
MICHELIA ALBA OIL,
MATE ABS,
MIMOSA PAYS ABS,
NARCISSUS ABS,
C6-12 ETHOXYLATED ALCOHOLS,
NEROLI EO,
OSMANTHUS ABS NAT,
PETITGRAIN ITALY EO,
PETITGRAIN S TERP,
ROSE ABS BULGARE,
TEA BLACK CEYLAN EXTR,
(+−)-2-propylheptanenitrile,
(2E)-2-METHYL-3-(4-METHYLPHENYL)-2-PROPEN-1-OL,
TUBEROSE INDIA ABS,
YLANG VOP,
1-(3-METHYL-1-BENZOFURAN-2-YL)ETHANONE,
1-(4-methylphenyl)ethenone,
methyl 2-aminobenzoate,
(+−)-(2,5-dimethyl-2,3-dihydro-1H-inden-2-yl)methanol,
2-METHOXYNAPHTHALENE,
(+−)-2-methyl-3-[4-(2-methyl-2-propanyl)phenyl]propanal,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
2-HEXYL-2-CYCLOPENTEN-1-ONE,
3-(4-TERT-BUTYLPHENYL)PROPANAL,
3,7-DIMETHYL-6-OCTEN-1-OL,
3,7-DIMETHYL-7-OCTEN-1-OL,
(+−)-2,4-dimethyl-4,4a,5,9b-tetrahydroindeno[1,2-d][1,3]dioxine,
ETHYL BENZOATE,
ETHYL PHENYLACETATE,
(+−)-(E)-3-METHYL-4-(2,6,6-TRIMETHYL-2-CYCLOHEXEN-1-YL)-3-BUTEN-2-ONE,
2-PHENYLETHANOL,
4-methyl-2-(2-methyl-1-propen-1-yl)tetrahydro-2H-pyran,
4-methyl-2-phenyltetrahydro-2H-pyran,
(3E)-4-(2,6,6-trimethyl-1-cyclohexen-1-yl)-3-buten-2-one,
(−)-(2S,4R)-4-methyl-2-(2-methyl-1-propen-1-yl)tetrahydro-2H-pyran,
1-(2-naphthyl)ethenone,
(4E)-4-methyl-5-(4-methylphenyl)-4-pentenal,
TRICYCLO[5.2.1.0(2,6)]DEC-3-EN-8-YL PROPANOATE,
3-methyl-2-[(2Z)-2-penten-1-yl]-2-cyclopenten-1-one,
(+−)-(3E)-4-(2,6,6-trimethyl-2-cyclohexen-1-yl)-3-buten-2-one,
(3E)-4-(2,6,6-trimethyl-1-cyclohexen-1-yl)-3-buten-2-one,
(1E)-1-(2,6,6-trimethyl-1-cyclohexen-1-yl)-1-penten-3-one,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
1-METHOXY-4-METHYLBENZENE,
(+−)-(3E)-3-methyl-4-(2,6,6-trimethyl-2-cyclohexen-1-yl)-3-buten-2-one,
(+−)-(1E)-1-(2,6,6-trimethyl-2-cyclohexen-1-yl)-1-penten-3-one,
(+−)-3-methyl-5-phenyl-1-pentanol,
(+−)-3,7-dimethyl-1,6-octadien-3-ol,
(+−)-(E)-3-METHYL-4-(2,6,6-TRIMETHYL-2-CYCLOHEXEN-1-YL)-3-BUTEN-2-ONE,
(E)-1-(2,6,6-TRIMETHYL-2-CYCLOHEXEN-1-YL)-1-PENTEN-3-ONE,
(+−)-(E)-1-(2,2-DIMETHYL-6-METHYLENE-1-CYCLOHEXYL)-1-PENTEN-3-ONE,
(E)-1-(2,6,6-TRIMETHYL-1-CYCLOHEXEN-1-YL)-1-PENTEN-3-ONE,
4-methyl-6-phenyl-2-hexanol,
3,7-DIMETHYL-2,6-OCTADIEN-1-OL,
(+−)-3-(1,3-BENZODIOXOL-5-YL)-2-METHYLPROPANAL,
(+−)-(E)-TRANS-alpha-IRONE,
(+−)-(E)-CIS-alpha-IRONE,
(+−)-(E)-beta-IRONE,
3,7-DIMETHYL-1,6-NONADIEN-3-OL,
methyl [3-oxo-2-pentylcyclopentyl]acetate,
2-PHENYLETHYL ACETATE,
2-PHENYLETHYL FORMATE,
(3Z)-3-hexen-1-yl salicylate,
1,1′-oxydibenzene,
tricyclo[5.2.1.0~2,6-]dec-3-en-8-yl acetate,
tricyclo[5.2.1.0~2,6-]dec-4-en-8-yl acetate,
(+−)-TRANS-3-METHYL-4-NONANOLIDE,
(+−)-3,7-DIMETHYL-3-OCTANOL,
(+−)-(Z)-7-DECEN-4-OLIDE,
(+−)-3,7-DIMETHYL-6-OCTEN-1-OL,
2-PHENYLETHYL PHENYLACETATE,
(+−)-5-ETHYL-2-NONANOL,
(+)-METHYL (1R)-CIS-3-OXO-2-PENTYL-1-CYCLOPENTANEACETATE,
1-octanol,
benzyl acetate,
CYCLOHEXYL 2-HYDROXYBENZOATE,
(+−)-8-DECEN-5-OLIDE,
CYCLOHEXYLIDENE(PHENYL)ACETONITRILE,
(+−)-3-(4-isopropylphenyl)-2-methylpropanal,
METHYL BENZOATE,
2-pentylcyclopentanol,
7-HYDROXY-3,7-DIMETHYLOCTANAL,
(+−)-7-hydroxy-3,7-dimethyloctanal,
(+−)-(3-methoxy-2-methylpropyl)benzene,
(+−)-3,7-dimethyl-1-octen-3-ol,
methyl cyclopentylideneacetate,
2-PHENYLETHYL 2-HYDROXYBENZOATE,
1-(5,6,7,8-tetrahydro-2-naphthalenyl)ethenone,
(+−)-2,2,2-TRICHLORO-1-PHENYLETHYL ACETATE,
methyl 2-((1 RS,2RS)-3-oxo-2-pentylcyclopentyl)acetate,
(2Z)-2-phenyl-2-hexenenitrile,
1,3-NONANEDIYL DIACETATE,
TETRAHYDRO-3-PENTYL-4(2H)-PYRANYL ACETATE,
(+−)-3,7-DIMETHYL-6-OCTENYL PROPANOATE,
(Z)-3,7-DIMETHYL-2,6-OCTADIEN-1-OL,
2-ETHOXYNAPHTHALENE,
(E)-2-PENTYL-3-PHENYL-2-PROPENAL,
9-DECEN-1-OL,
2-PHENYLETHYL ISOBUTYRATE,
benzyl salicylate,
4-CYCLOHEXYL-2-METHYL-2-BUTANOL,
1-NONANOL,
3-METHYLBUTYL 2-PHENYLETHYL ETHER,
(+−)-3,7-dimethyl-3-octanyl acetate,
ISOBUTYL PHENYLACETATE,
methyl {(1RS,2RS)-3-oxo-2-[(2Z)-2-penten-1-yl]cyclopentyl}acetate,
(+−)-TETRAHYDRO-2-ISOBUTYL-4-METHYL-4(2H)-PYRANOL,
ISOBUTYL 2-HYDROXYBENZOATE,
(−)-(2S)-1-oxo-1-(2-propanyloxy)-2-propanyl 2,2-dimethylpropanoate,
1-dodecanol,
(+−)-2-phenyl-1-propanol,
2-METHYLBUTYL SALICYLATE,
PENTYL SALICYLATE,
3-METHYLBUTYL PHENYLACETATE,
2-METHYLBUTYL PHENYLACETATE,
3-pentyltetrahydro-2H-pyran-4-yl acetate,
1,1-DIMETHYL-2-PHENYLETHYL ACETATE,
3,7-DIMETHYL-6-OCTENYL ACETATE,
BENZYL PHENYLACETATE,
[trans-4-(2-propanyl)cyclohexyl]methanol,
[cis-4-(2-propanyl)cyclohexyl]methanol,
(2E)-2-benzylideneoctanal,
2-BENZYL-4,4,6-TRIMETHYL-1,3-DIOXANE,
3-[(1R)-4-methyl-3-cyclohexen-1-yl]-1-butanol,
CIS-2-BENZYL-1,3-DIOXAN-5-OL,
2-BENZYL-1,3-DIOXOLANE-4-METHANOL,
TRANS-2-BENZYL-1,3-DIOXAN-5-OL,
6,10-dimethyl-5,9-undecadien-2-one,
HEXYL 2-HYDROXYBENZOATE,
3,7,11-TRIMETHYL-1,6,10-DODECATRIEN-3-OL,
2-phenoxyethanol,
1-PHENYL-1-ETHANOL,
(E)-3,7-DIMETHYL-2,6-OCTADIENYL PROPANOATE,
2-METHYL-4-PHENYL-2-BUTANOL,
(+−)-2,4,6-TRIMETHYL-3-CYCLOHEXENE-1-METHANOL,
2-METHYL-1-PHENYL-2-PROPANOL,
2-phenoxyethyl 2-methylpropanoate,
decyl acetate,
(E)-3,7-dimethyl-2,6-octadienyl acetate,
(2Z)-3,7-dimethyl-2,6-octadien-1-yl acetate,
(+−)-3,7-dimethyloctyl acetate,
tricyclo[5.2.1.0~2,6-]dec-3-en-8-yl acetate,
tricyclo[5.2.1.0~2,6-]dec-4-en-8-yl acetate,
citronellyl acetate,
neryl acetate,
geranyl acetate,
3,5,5-trimethylhexyl acetate,
4-phenyl-2-butanone,
4-(2,6,6-trimethyl-1-cyclohexen-1-yl)-2-butanone,
(3E)-4-(2,6,6-trimethyl-1-cyclohexen-1-yl)-3-buten-2-one,
4-(1,3-benzodioxol-5-yl)-2-butanone,
CAPUCINE M SA,
CASSIE ABSOLUTE,
1-phenyl-2-(1-propoxyethoxy)ethane,
(+−)-8,8-dimethyl-1,2,3,4,5,6,7,8-octahydro-2-naphthalenecarbaldehyde,
(+−)-5,5-dimethyl-1,2,3,4,5,6,7,8-octahydro-2-naphthalenecarbaldehyde,
(+−)-2,2-dimethyltricyclo[6.2.1.0~1,6-]undecan-7-one,
(+−)-2-pentylcyclopentanone,
2,6-dimethyl-2-heptanol,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
(+−)-(4Z)-4,8-dimethyl-4,9-decadienal,
(+−)-(4E)-4,8-dimethyl-4,9-decadienal,
(+−)-3-(3-isopropyl-1-phenyl)butanal,
(+−)-3,7-dimethyl-6-octenyl formate,
(+−)-3,7-dimethyl-6-octen-1-yl formate,
(2E)-3,7-dimethyl-2,6-octadien-1-yl formate,
GARDENIA FIRABSOLUE,
thiodamascone,
(1Z)-2-methyl-1-undecen-1-yl 2-phenylethyl ether,
(1E)-2-methyl-1-undecen-1-yl 2-phenylethyl ether,
thioionone,
(+−)-4-(dodecylthio)-4-(2,6,6-trimethyl-2-cyclohexen-1-yl)-2-butanone,
(+−)-4-(dodecylthio)-4-(2,6,6-trimethyl-1-cyclohexen-1-yl)-2-butanone,
benzo[d][1,3]dioxole-5-carbaldehyde,
(+−)-(3E)-4-(2,6,6-trimethyl-2-cyclohexen-1-yl)-3-buten-2-one,
2-nonenenitrile,
cis-4-(2-methyl-2-propanyl)cyclohexyl acetate,
trans-4-(2-methyl-2-propanyl)cyclohexyl acetate,
MUGUET FIRBEST,
(+−)-(4Z)-5-cyclohexyl-2,4-dimethyl-4-pentenal,
(+−)-(4E)-5-cyclohexyl-2,4-dimethyl-4-pentenal,
ORENOL P SAD,
PALMAROSA,
PIVOINE BLANCHE FIRBEST,
benzyl propanoate,
(+−)-methyl 2,2-dimethyl-6-methylidenecyclohexanecarboxylate,
(−)-(3R)-3,7-dimethyl-1,6-octadien-3-ol,
(+−)-3,7-dimethyl-1-octanol,
TEA GREEN FIRBEST,
3-(4,4-dimethyl-1-cyclohexen-1-yl)propanal,
LINDEN FIRBEST,
VIOTRIL BASE,
PETITGRAIN PARAGUAY HE,
YLANG EXTRA INFUSION,
YLANG EXTRA FIRNAT,
3-[4-(2-hydroxy-2-methylpropyl)phenyl]propanal,
(+−)-2-[4-(2-hydroxy-2-methylpropyl)phenyl]propanal,
3-(3-(2-hydroxy-2-methylpropyl)phenyl)propanal,
and any combination thereof.

14. A consumer product comprising the mental performance enhancing composition according to claim 13.

15. The consumer product according to claim 14, wherein the consumer product is a perfume, a fabric care product, a body-care product, a cosmetic preparation, a skin-care product, an air care product, or a home care product.

16. Non-therapeutic use of the mental performance enhancing composition according to claim 13 or consumer product according to any one of claims 14 or 15 to enhance the mental performance of a subject, typically a human being.

17. A method for evaluating the ability of a material to deliver a consumer benefit over time, the method comprising conducting one or more sessions, wherein each session comprises:
a.) Exposing the subject to a material having an odor,
b.) Capturing the subject's perception of odor intensity,
c.) Optionally, allowing the subject to perform one or more tasks and measuring a parameter associated with each task,
d.) capturing the subject's mood or mental state,
e.) repeating steps a. to d. 1 to 10, typically 1 to 5, times.

18. The method according to claim 17, wherein the consumer benefit is an emotion, mood, feeling, mental state, or enhancement thereof, typically enhancement of mental performance.

19. The method according to claim 17 or 18, wherein the method comprises conducting two or more sessions, typically three or more sessions, wherein in at least one session, the subject is exposed to a material having no odor.

20. The method according to any one of claims 17 to 19, wherein each session is conducted 1 to 20 days, typically 1 to 15 days, more typically 1 to 10 days, apart.

21. The method according to any one of claims 17 to 20, wherein each repetition of steps a-d is 1 to 60 minutes, typically 1 to 30 minutes, more typically 1 to 20 minutes, from the previous repetition.

22. The method according to any one of claims 17 to 21, wherein each session is at least 30 minutes, typically at least 1 hour, more typically at least 2 hours.

23. The method according to any one of claims 17 to 22, wherein the task is a Stroop task or an arithmetic task.

24. The method according to any one of claims 17 to 23, wherein the parameter associated with each task is response time or accuracy.
